(19) United States
(12) Patent Application Publication
(10) Pub. No.: US 2023/0332210 A1
(43) Pub. Date: Oct. 19, 2023

(54) REPORTER AND USE THEREOF

(71) Applicant: SFC CO., LTD., Cheongju-si (KR)

(72) Inventors: Do Min LEE, Cheongju-si (KR); Ho Young SI, Cheongju-si (KR); Jae Hyung JO, Cheongju-si (KR); Goutam MASANTA, Cheongju-si (KR); Ju Man SONG, Cheongju-si (KR); Min Su EUM, Cheongju-si (KR); Jong Tae JE, Cheongju-si (KR)

(73) Assignee: SFC CO., LTD., Cheongju-si (KR)

(21) Appl. No.: 18/338,178

(22) Filed: Jun. 20, 2023

Related U.S. Application Data
(63) Continuation of application No. PCT/KR2021/018869, filed on Dec. 13, 2021.

(30) Foreign Application Priority Data
Dec. 21, 2020 (KR) 10-2020-0179829
Dec. 9, 2021 (KR) 10-2021-0175655

(51) Int. Cl. C12Q 1/6818; C09K 11/06; C09B 67/00

(52) U.S. Cl. CPC C12Q 1/6818; C09K 11/06; C09B 67/0001; C09K 2211/1022; C12Q 1/686

(57) ABSTRACT

The present invention relates to a fluorescent reporter exhibiting luminescence at an excited energy level as well as labeling a nucleic acid including DNA, and various uses thereof.

### CROSS-REFERENCE TO RELATED APPLICATION

This application is based on the PCT Application No. PCT/KR2021/018869, filed on Dec. 13, 2021, and claims the benefit of priority from Korean Patent Application No. 10-2020-0179829, filed on Dec. 21, 2020, and Korean Patent Application No. 10-2021-0175655, filed on Dec. 9, 2021, the disclosures of which are incorporated herein by reference in its entirety.

### BACKGROUND

### 1. Field of the Invention

The present invention relates to a fluorescent reporter exhibiting luminescence at an excited energy level as well as labeling a nucleic acid including DNA, and various uses thereof.

### 2. Discussion of Related Art

In the field of biotechnology, fluorescent dyes are used as a means for visualizing biological phenomena to observe these phenomena at the cell level in vivo or in vitro, or to execute bio-imaging and examine a disease area.

While there are biomolecules self-emitting light, such as a green fluorescent protein (GFP), generally, after bio tissue or cells and biomolecules at a lower level are stained with fluorescent dyes, or biomolecules including proteins, nucleic acids, etc. are labeled with fluorescent dyes, imaging data is obtained with various techniques using optical equipment capable of detecting fluorescent signals.

As optical analysis equipment mainly used, in addition to equipment for research for observing cells, such as a fluorescence microscope, a confocal microscope, flow cytometry, a microarray, a quantitative PCR system, nucleic acid and protein isolation, an electrophoresis apparatus for analysis, or a real-time in vivo imaging system, equipment for diagnosis and treatment such as in vitro diagnosis equipment based on an immune assay or nucleic acid and protein diagnosis kit (or biochip), combining PCR analysis and statistical techniques, and an operating table and endoscopic equipment for image-guided surgery is known, and new applications and equipment with a higher level of resolution and data processing capability are constantly being developed.

To apply fluorescent dyes in a biology field, generally, when being present in a medium in which most biomolecules are present, that is, an aqueous solution, fluorescent dyes preferably have less photo bleaching and quenching, a high molecular extinction coefficient, and high quantum efficiency, and are stable under various pH conditions.

While various fluorescent dyes have been used in various research fields, those that satisfy all of the above conditions are rare in a biology field, and representative structures of fluorescent dyes, which are currently used, include coumarin, cyanine, BODIPY, fluorescein, rhodamine, pyrene, carbopyronine, oxazine, xanthene, thioxanthene, and acridine. Among them, particularly, rhodamine derivatives and polymethine-based cyanine derivatives are main structures.

Particularly, cyanine-based fluorescent dyes are linked to both ends of polymethine via a nitrogen-containing hetero ring, and have not only structural advantages of realizing various colors of fluorescence throughout the entire visible and near-infrared ranges from 450 to 800 nm but also generally have an optical property of a very high extinction coefficient by adjusting a polymethine length.

The background art relating to the present invention is disclosed in Korean Unexamined Patent Application Publication No. 10-2017-0026245 (published on Mar. 8, 2017), and in this document, a perylene-based compound, a method of preparing the same, and a fluorescent dye including the same are described.

### SUMMARY OF THE INVENTION

The present invention is directed to providing a novel reporter as a compound that can be used widely to identify a biomolecule in an optical imaging field.

The present invention is also directed to providing various uses of the novel reported defined herein, such as an oligonucleotide for detecting a nucleic acid, a composition for detecting a nucleic acid, a support for detecting a nucleic acid, including the novel reporter, and a method of detecting a nucleic acid.

According to one aspect of the present invention for solving the above technical problems, a reporter represented by Formula 1 or 2 below is provided.

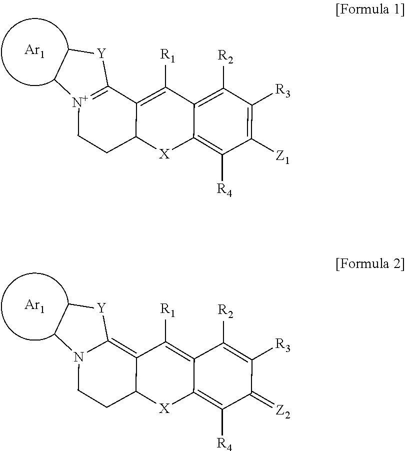

Here,

Ar₁ is substituted or unsubstituted C₆-C₂₀ aryl or C₆-C₂₀ heteroaryl including at least one hetero atom,

Z₁ is NR₅R₆ or OR₇,

Z₂ is NR₈ or O,

X is O or S,

Y is CR₉R₁₀, NR₁₁, O or S,

R₁ to R₁₁ are each independently a moiety selected from hydrogen, deuterium, substituted or unsubstituted C₁-C₁₀ alkyl, substituted or unsubstituted C₁-C₁₀ heteroalkyl including at least one hetero atom, substituted or unsubstituted C₂-C₁₀ alkenyl, substituted or unsubstituted C₂-C₁₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O—), substituted or unsubstituted C₁-C₁₀ alkoxy, substituted or unsubstituted C₃-C₂₀ cycloalkyloxy, substituted or unsubstituted C₅-C₂₀ aryloxy, substituted or unsubstituted C₂-C₂₀ heteroaryloxy, substituted or unsubstituted C₅-C₂₀ aryl, substituted or unsubstituted C₂-C₂₀ heteroaryl, substituted or unsubstituted C₅-C₂₀ aralkyl, substituted or unsubstituted C₁-C₁₀ alkylthio, substituted or unsubstituted C₅-C₂₀ arylthio, substituted or unsubstituted C₃-C₂₀ cycloalkylthio, substituted or unsubstituted C₂-C₂₀ heteroarylthio, substituted or unsubstituted acylamino, acyloxy, substituted or unsubstituted phosphino, carboxylate (—CO₂—), trifluoromethylsulfonyl (—SO₂CF₃), substituted or unsubstituted ammonium, nitro, sulfonic acid (—SO₃H), sulfonate, substituted sulfonyl, substituted sulfonic acid ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkyleneoxide, or each independently forms a ring by binding adjacent two moieties to each other, and

at least one of R₁ to R₁₁ is a moiety substituted with phosphoramidite or active ester.

In addition, the reporter may be represented by Formula 5 or 6 below.

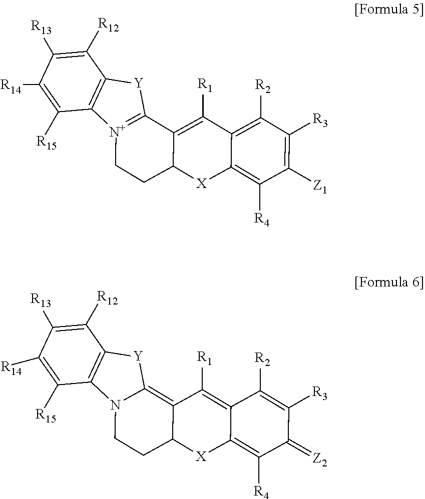

Here,

Z₁ is NR₅R₆ or OR₇,

Z₂ is NR₈ or O,

X is O or S,

Y is CR₉R₁₀, NR₁₁, O or S,

R₁ to R₁₅ are each independently a moiety selected from hydrogen, deuterium, substituted or unsubstituted C₁-C₁₀ alkyl, substituted or unsubstituted C₁-C₁₀ heteroalkyl including at least one hetero atom, substituted or unsubstituted C₂-C₁₀ alkenyl, substituted or unsubstituted C₂-C₁₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O—), substituted or unsubstituted C₁-C₁₀ alkoxy, substituted or unsubstituted C₃-C₂₀ cycloalkyloxy, substituted or unsubstituted C₅-C₂₀ aryloxy, substituted or unsubstituted C₂-C₂₀ heteroaryloxy, substituted or unsubstituted C₅-C₂₀ aryl, substituted or unsubstituted C₂-C₂₀ heteroaryl, substituted or unsubstituted C₅-C₂₀ aralkyl, substituted or unsubstituted C₁-C₁₀ alkylthio, substituted or unsubstituted C₅-C₂₀ arylthio, substituted or unsubstituted C₃-C₂₀ cycloalkylthio, substituted or unsubstituted C₂-C₂₀ heteroarylthio, substituted or unsubstituted acylamino, acyloxy, substituted or unsubstituted phosphino, carboxylate (—CO₂—), trifluoromethylsulfonyl (—SO₂CF₃), substituted or unsubstituted ammonium, nitro, sulfonic acid (—SO₃H), sulfonate, substituted sulfonyl, substituted sulfonic acid ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkyleneoxide, or each independently forms a ring by binding adjacent two moieties to each other,

at least one of R₁ to R₁₅ is a moiety substituted with phosphoramidite or active ester.

In addition, according to another aspect of the present invention, an oligonucleotide including the reporter defined herein and a quencher is provided.

In addition, according to still another aspect of the present invention, a composition for detecting a nucleic acid, which includes the oligonucleotide defined herein is provided.

In addition, according to yet another aspect of the present invention, a support for detecting a nucleic acid, which includes the reporter defined herein or a probe dual-labeled with a reporter and a quencher; a support; and a linker connecting the reporter or the probe with the support, is provided.

In addition, according to yet another aspect of the present invention, a method of detecting a nucleic acid, which includes: (a) preparing a reaction mixture including a target nucleic acid, a reagent necessary for amplifying the target nucleic acid, and the oligonucleotide defined herein, (b) amplifying the target nucleic acid in the reaction mixture by a polymerase chain reaction, and (c) measuring a fluorescence intensity of the reaction mixture, is provided.

A reporter according to the present invention can be effectively used to label various targets including a biomolecule due to excellent solubility in water and an excellent fluorescence intensity.

### BRIEF DESCRIPTION OF THE DRAWINGS

FIG. 1 is a graph showing the result of real-time PCR repeated twice using dual-labeled probes according to Example 1, Comparative Examples 1 to 3 for a black queen cell virus (BQCV) target.

FIG. 2 shows average Ct values of real-time PCR repeated twice using dual-labeled probes according to Example 1, Comparative Examples 1 to 3 for BQCV target.

FIG. 3 shows average RFU values of real-time PCR repeated twice using dual-labeled probes according to Example 1, Comparative Examples 1 to 3 for BQCV target.

FIG. 4 is a graph showing the result of real-time PCR repeated twice using dual-labeled probes according to Example 2, Comparative Examples 1 to 3 for a Chlamydia trachomatis (CT) target.

FIG. 5 shows average Ct values of real-time PCR repeated twice using dual-labeled probes according to Example 2, Comparative Examples 1 to 3 for a CT target.

FIG. 6 shows average RFU values of real-time PCR repeated twice using dual-labeled probes according to Example 2, Comparative Examples 1 to 3 for a CT target.

### DETAILED DESCRIPTION OF EXEMPLARY EMBODIMENTS

In order to better understand the present invention, certain terms are defined herein for convenience. Unless defined otherwise herein, scientific and technical terms used herein will have meanings commonly understood by those of ordinary skill in the art.

In addition, unless specifically indicated otherwise, terms in a singular form also include plural forms, and terms in a plural form may also include singular forms as well.

Novel Reporter for Labeling Nucleic Acid

According to one aspect of the present invention, a reporter for labeling a nucleic acid, represented by Formula 1 or 2 below, is provided.

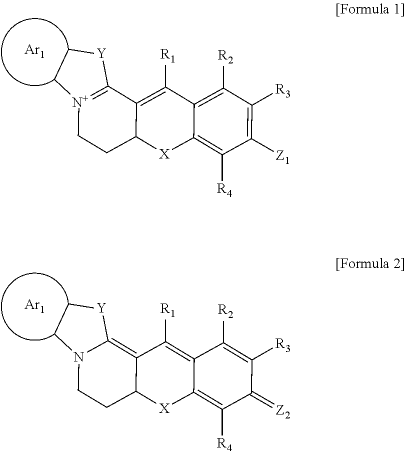

Here,

Ar₁ is substituted or unsubstituted C₆-C₂₀ aryl or C₆-C₂₀ heteroaryl including at least one hetero atom,

Z₁ is NR₅R₆ or OR₇,

Z₂ is NR₈ or O,

X is O or S,

Y is CR₉R₁₀, NR₁₁, O or S,

R₁ to R₁₁ are each independently a moiety selected from hydrogen, deuterium, substituted or unsubstituted C₁-C₁₀ alkyl, substituted or unsubstituted C₁-C₁₀ heteroalkyl including at least one hetero atom, substituted or unsubstituted C₂-C₁₀ alkenyl, substituted or unsubstituted C₂-C₁₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O—), substituted or unsubstituted C₁-C₁₀ alkoxy, substituted or unsubstituted C₃-C₂₀ cycloalkyloxy, substituted or unsubstituted C₅-C₂₀ aryloxy, substituted or unsubstituted C₂-C₂₀ heteroaryloxy, substituted or unsubstituted C₅-C₂₀ aryl, substituted or unsubstituted C₂-C₂₀ heteroaryl, substituted or unsubstituted C₅-C₂₀ aralkyl, substituted or unsubstituted C₁-C₁₀ alkylthio, substituted or unsubstituted C₅-C₂₀ arylthio, substituted or unsubstituted C₃-C₂₀ cycloalkylthio, substituted or unsubstituted C₂-C₂₀ heteroarylthio, substituted or unsubstituted acylamino, acyloxy, substituted or unsubstituted phosphino, carboxylate (—CO₂—), trifluoromethylsulfonyl (—SO₂CF₃), substituted or unsubstituted ammonium, nitro, sulfonic acid (—SO₃H), sulfonate, substituted sulfonyl, substituted sulfonic acid ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkyleneoxide, or each independently forms a ring by binding adjacent two moieties to each other.

Here, at least one of R₁ to R₁₁ is a moiety substituted with phosphoramidite or active ester.

In addition, when any moiety of R₁ to R₁₁ is a substituted moiety, at least one carbon in the moiety may be substituted with (1) any moiety selected from deuterium, substituted or unsubstituted C₁-C₁₀ alkyl, substituted or unsubstituted C₁-C₁₀ heteroalkyl including at least one hetero atom, substituted or unsubstituted C₂-C₁₀ alkenyl, substituted or unsubstituted C₂-C₁₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O—), substituted or unsubstituted C₁-C₁₀ alkoxy, substituted or unsubstituted C₃-C₂₀ cycloalkyloxy, substituted or unsubstituted C₅-C₂₀ aryloxy, substituted or unsubstituted C₂-C₂₀ heteroaryloxy, substituted or unsubstituted C₅-C₂₀ aryl, substituted or unsubstituted C₂-C₂₀ heteroaryl, substituted or unsubstituted C₅-C₂₀ aralkyl, substituted or unsubstituted C₁-C₁₀ alkylthio, substituted or unsubstituted C₅-C₂₀ arylthio, substituted or unsubstituted C₃-C₂₀ cycloalkylthio, substituted or unsubstituted C₂-C₂₀ heteroarylthio, substituted or unsubstituted acylamino, acyloxy, substituted or unsubstituted phosphino, carboxylate (—CO₂—), trifluoromethylsulfonyl (—SO₂CF₃), substituted or unsubstituted ammonium, nitro, sulfonic acid (—SO₃H), sulfonate, substituted sulfonyl, substituted sulfonic acid ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkyleneoxide, or (2) phosphoramidite or active ester.

Reporters according to various embodiments of the present invention can be labeled by binding with a target biomolecule (e.g., a nucleic acid) via a moiety substituted with phosphoramidite or active ester present in at least one of R₁ to R₁₁ for labeling.

Here, at least one of R₁ to R₁₁ may be a moiety in which an end is substituted with phosphoramidite or active ester.

The active ester may be selected from a N-hydroxy succinimide derivative, a hydroxybenzotriazole derivative, a 1-hydroxy-7-azabenzotriazole derivative, a nitrophenol derivative, and a pentafluorophenol derivative.

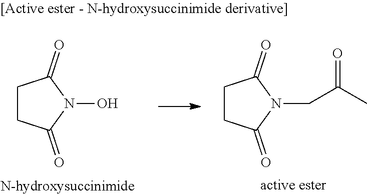

Here, * indicates a position to which the moiety binds.

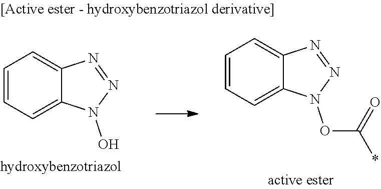 [SMILES: On1nnc2ccccc21 ; *C(=O)On1nnc2ccccc21]

Here, * indicates a position to which the moiety binds.

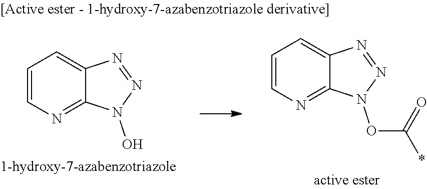 [SMILES: On1nnc2cccnc21 ; *C(=O)On1nnc2cccnc21]

Here, * indicates a position to which the moiety binds.

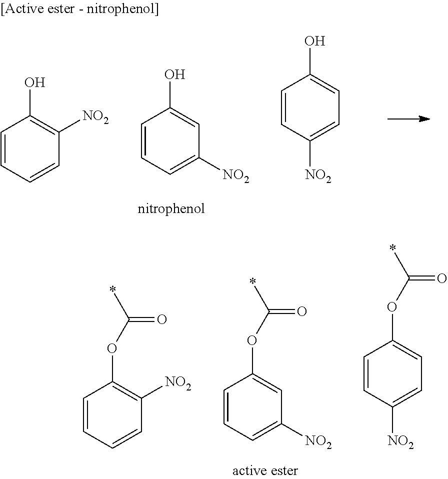 [SMILES: O=[N+]([O-])c1ccccc1O ; O=[N+]([O-])c1cccc(O)c1 ; O=[N+]([O-])c1ccc(O)cc1 ; *C(=O)Oc1ccccc1[N+](=O)[O-] ; *C(=O)Oc1cccc([N+](=O)[O-])c1 ; *C(=O)Oc1ccc([N+](=O)[O-])cc1]

Here, * indicates a position to which the moiety binds.

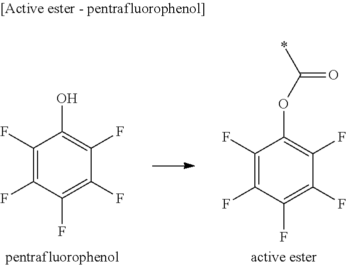 [SMILES: Oc1c(F)c(F)c(F)c(F)c1F ; *C(=O)Oc1c(F)c(F)c(F)c(F)c1F]

In another embodiment, at least one of R₁ to R₁₁ may be any moiety substituted with a moiety represented by Formula 3 below.

Here, * indicates a position to which the moiety binds.

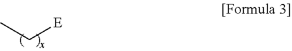

Here, * indicates a position to which the moiety binds, x is an integer between 2 to 10, and E is phosphoramidite or active ester.

When an end of the moiety represented by Formula 3 is phosphoramidite, the moiety may be represented by Formula 3-1 below.

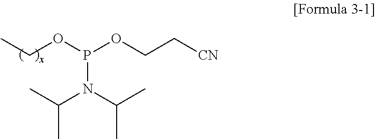

In still another embodiment, at least one of R₁ to R₁₁ may be any moiety substituted with a moiety represented by Formula 4 below.

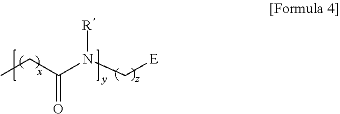

Here, * indicates a position to which the moiety binds, x is an integer between 2 to 10, y is an integer between 1 to 5, z is an integer between 0 to 5, R′ is hydrogen, deuterium, or substituted or unsubstituted C₁-C₁₀ alkyl, and E is phosphoramidite or active ester.

When an end of the moiety represented by Formula 4 is phosphoramidite, the moiety may be represented by Formula 4-1 below.

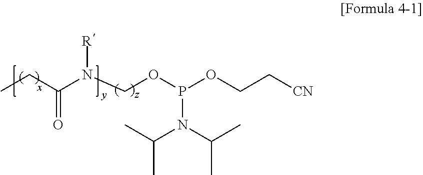

While R₁ to R₁₁ may each be independently present as the moieties defined above, in some embodiments, at least one of R₁ to R₁₁ may be bound with an adjacent substituent, thereby forming a substituted or unsubstituted ring (e.g., a four-element ring, a five-element ring, a six-element ring, a ring consisting of more than 6 atoms, or a fused ring in which multiple rings are joined). In addition, the ring may be an aliphatic or aromatic ring, and may include at least one hetero atom.

When at least one of R₁ to R₁₁ is bound with an adjacent substituent and thus forms a substituted or unsubstituted ring, at least one of R₁ to R₁₁ may be bound with an adjacent substituent via C, N, O, S, Se or Si, or may be directly bound with an adjacent substituent by a single bond.

In one embodiment, the reporter is represented by Formula 1, and when Z₁ is NR₅R₆, R₅ or R₆ may be bound with R₃ or R₄, thereby forming a substituted or unsubstituted ring.

The rings unsubstituted or substituted with R₅ and R₆ are formed independently from each other, and in one compound, a ring formed by bonding R₅ and R₃ (or R₄) with each other and a ring formed by bonding R₆ and R₄ (or R₃) may be present at the same time.

In another embodiment, the reporter may be represented by Formula 1, and when Z₁ is OR₇, R₇ may be bound with R₃ or R₄, thereby forming a substituted or unsubstituted ring.

In still another embodiment, the reporter may be represented by Formula 2, and when Z₂ is NR₈, R₈ may be bound with R₃ or R₄, thereby forming a substituted or unsubstituted ring.

When at least one of R₁ to R₁₁ is bound with an adjacent substituent to form a substituted ring, at least one carbon in the ring may be substituted with (1) any moiety selected from deuterium, substituted or unsubstituted C₁-C₁₀ alkyl, substituted or unsubstituted C₁-C₁₀ heteroalkyl including at least one hetero atom, substituted or unsubstituted C₂-C₁₀ alkenyl, substituted or unsubstituted C₂-C₁₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O—), substituted or unsubstituted C₁-C₁₀ alkoxy, substituted or unsubstituted C₃-C₂₀ cycloalkyloxy, substituted or unsubstituted C₅-C₂₀ aryloxy, substituted or unsubstituted C₂-C₂₀ heteroaryloxy, substituted or unsubstituted C₅-C₂₀ aryl, substituted or unsubstituted C₂-C₂₀ heteroaryl, substituted or unsubstituted C₅-C₂₀ aralkyl, substituted or unsubstituted C₁-C₁₀ alkylthio, substituted or unsubstituted C₅-C₂₀ arylthio, substituted or unsubstituted C₃-C₂₀ cycloalkylthio, substituted or unsubstituted C₂-C₂₀ heteroarylthio, substituted or unsubstituted acylamino, acyloxy, substituted or unsubstituted phosphino, carboxylate (—CO₂—), trifluoromethylsulfonyl (—SO₂CF₃), substituted or unsubstituted ammonium, nitro, sulfonic acid (—SO₃H), sulfonate, substituted sulfonyl, substituted sulfonic acid ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkyleneoxide, or (2) phosphoramidite or active ester.

In addition, a reporter for labeling a nucleic acid according to the present invention may be represented by Formula 5 or 6 below. Unless defined otherwise, the reporter for labeling a nucleic acid represented by Formula 5 or 6 below is the same within a range overlapping with the reporter for labeling a nucleic acid represented by Formula 1 or 2.

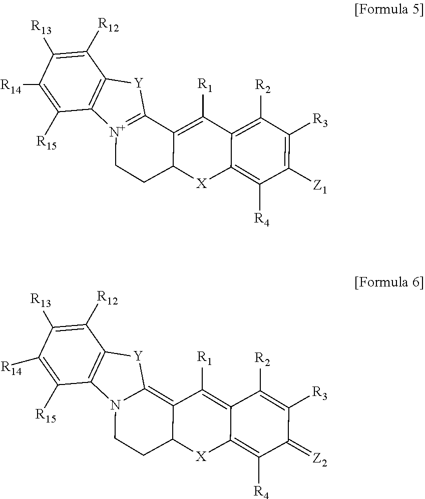

Here,

Z₁ is NR₅R₆ or OR₇,

Z₂ is NR₈ or O,

X is O or S,

Y is CR₉R₁₀, NR₁₁, O or S,

R₁ to R₁₅ are each independently a moiety selected from hydrogen, deuterium, substituted or unsubstituted C₁-C₁₀ alkyl, substituted or unsubstituted C₁-C₁₀ heteroalkyl including at least one hetero atom, substituted or unsubstituted C₂-C₁₀ alkenyl, substituted or unsubstituted C₂-C₁₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O—), substituted or unsubstituted C₁-C₁₀ alkoxy, substituted or unsubstituted C₃-C₂₀ cycloalkyloxy, substituted or unsubstituted C₅-C₂₀ aryloxy, substituted or unsubstituted C₂-C₂₀ heteroaryloxy, substituted or unsubstituted C₅-C₂₀ aryl, substituted or unsubstituted C₂-C₂₀ heteroaryl, substituted or unsubstituted C₅-C₂₀ aralkyl, substituted or unsubstituted C₁-C₁₀ alkylthio, substituted or unsubstituted C₅-C₂₀ arylthio, substituted or unsubstituted C₃-C₂₀ cycloalkylthio, substituted or unsubstituted C₂-C₂₀ heteroarylthio, substituted or unsubstituted acylamino, acyloxy, substituted or unsubstituted phosphino, carboxylate (—CO₂—), trifluoromethylsulfonyl (—SO₂CF₃), substituted or unsubstituted ammonium, nitro, sulfonic acid (—SO₃H), sulfonate, substituted sulfonyl, substituted sulfonic acid ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkyleneoxide, or each independently forms a ring by binding adjacent two moieties to each other.

Here, at least one of R₁ to R₁₅ is a moiety substituted with phosphoramidite or active ester.

In addition, when any moiety of R₁ to R₁₅ is a substituted moiety, at least one carbon of the moiety may be substituted with (1) any moiety selected from deuterium, substituted or unsubstituted C₁-C₁₀ alkyl, substituted or unsubstituted C₁-C₁₀ heteroalkyl including at least one hetero atom, substituted or unsubstituted C₂-C₁₀ alkenyl, substituted or unsubstituted C₂-C₁₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O—), substituted or unsubstituted C₁-C₁₀ alkoxy, substituted or unsubstituted C₃-C₂₀ cycloalkyloxy, substituted or unsubstituted C₅-C₂₀ aryloxy, substituted or unsubstituted C₂-C₂₀ heteroaryloxy, substituted or unsubstituted C₅-C₂₀ aryl, substituted or unsubstituted C₂-C₂₀ heteroaryl, substituted or unsubstituted C₅-C₂₀ aralkyl, substituted or unsubstituted C₁-C₁₀ alkylthio, substituted or unsubstituted C₅-C₂₀ arylthio, substituted or unsubstituted C₃-C₂₀ cycloalkylthio, substituted or unsubstituted C₂-C₂₀ heteroarylthio, substituted or unsubstituted acylamino, acyloxy, substituted or unsubstituted phosphino, carboxylate (—CO₂—), trifluoromethylsulfonyl (—SO₂CF₃), substituted or unsubstituted ammonium, nitro, sulfonic acid (—SO₃H), sulfonate, substituted sulfonyl, substituted sulfonic acid ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkyleneoxide, or (2) phosphoramidite or active ester.

Reporters according to various embodiments of the present invention can be labeled by bonding with a target biomolecule (e.g., a nucleic acid) via a moiety substituted with phosphoramidite or active ester present in at least one of R₁ to R₁₅.

Here, at least one of R₁ to R₁₅ may be a moiety in which an end is substituted with phosphoramidite or active ester.

While R₁ to R₁₅ may each be independently present as moieties defined herein, in some embodiments, at least one of R₁ to R₁₅ may be bound with an adjacent substituent, thereby forming a substituted or unsubstituted ring (e.g., a four-element ring, a five-element ring, a six-element ring or a ring consisting of more than 6 elements, or a fusion ring in which a plurality of rings are conjugated). In addition, the ring may be an aliphatic or aromatic ring.

When at least one of R₁ to R₁₅ is bound with an adjacent substituent to form a substituted or unsubstituted ring, at least one of R₁ to R₁₅ may be bound with an adjacent substituent via C, N, O, S, Se or Si, or may be directly bound with an adjacent substituent by a single bond.

In addition, in one embodiment, two adjacent substituents of R₁₂ to R₁₅ may be bound with each other to form a substituted or unsubstituted ring.

Formation of the substituted or unsubstituted ring by binding two adjacent substituents of R₁₂ to R₁₅ is independent of each other, and for example, a ring formed by bonding R₁₂ and R₁₃ with each other and a ring formed by bonding R₁₄ and R₁₅ with each other may exist simultaneously in one compound.

When at least one of R₁ to R₁₅ is bound with an adjacent substituent to form a substituted ring, at least one carbon in the ring may be substituted with (1) any moiety selected from deuterium, substituted or unsubstituted C₁-C₁₀ alkyl, substituted or unsubstituted C₁-C₁₀ heteroalkyl including at least one hetero atom, substituted or unsubstituted C₂-C₁₀ alkenyl, substituted or unsubstituted C₂-C₁₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O—), substituted or unsubstituted C₁-C₁₀ alkoxy, substituted or unsubstituted C₃-C₂₀ cycloalkyloxy, substituted or unsubstituted C₅-C₂₀ aryloxy, substituted or unsubstituted C₂-C₂₀ heteroaryloxy, substituted or unsubstituted C₅-C₂₀ aryl, substituted or unsubstituted C₂-C₂₀ heteroaryl, substituted or unsubstituted C₅-C₂₀ aralkyl, substituted or unsubstituted C₁-C₁₀ alkylthio, substituted or unsubstituted C₅-C₂₀ arylthio, substituted or unsubstituted C₃-C₂₀ cycloalkylthio, substituted or unsubstituted C₂-C₂₀ heteroarylthio, substituted or unsubstituted acylamino, acyloxy, substituted or unsubstituted phosphino, carboxylate (—CO₂—), trifluoromethylsulfonyl (—SO₂CF₃), substituted or unsubstituted ammonium, nitro, sulfonic acid (—SO₃H), sulfonate, substituted sulfonyl, substituted sulfonic acid ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkyleneoxide, or (2) phosphoramidite or active ester.

In the present invention, when Rₐ (a is a natural number between 1 to 14) is alkenyl or alkynyl, sp²-hybrid carbon of alkenyl or sp-hybrid carbon of alkynyl may be directly bound, or indirectly bound by sp³-hybrid carbon of alkyl binding to sp²-hybrid carbon of alkenyl or sp-hybrid carbon of alkynyl.

In the present invention, a Cₐ-C_{b} moiety is a moiety that has a to b carbon atoms. For example, Cₐ-C_{b} alkyl is a saturated aliphatic group, including linear and branched alkyls having a to b carbon atoms. The linear or branched alkyls may have 40 or less carbon atoms (e.g., C₁-C₁₀ linear or C₃-C₁₀ branched) in the main chain thereof.

Specifically, the alkyl may be methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, n-heptyl, or n-octyl.

In addition, in the present invention, alkoxy is either of an —O-(alkyl) group and an —O-(unsubstituted cycloalkyl) group, and is linear or branched hydrocarbon having one or more ether groups and 1 to 10 carbon atoms.

Specific examples of the alkoxy include methoxy, ethoxy, propoxy, isopropoxy, n-butoxy, tert-butoxy, sec-butoxy, n-pentoxy, n-hexoxy, 1,2-dimethylbutoxy, cyclopropyloxy, cyclobutyloxy, cyclopentyloxy, and cyclohexyloxy, but the present invention is not limited thereto.

In addition, in the present invention, halogen is fluoro (—F), chloro (—Cl), bromo (—Br), or iodo (—I), and haloalkyl is alkyl substituted with the above-described halogen.

For example, halomethyl is methyl in which at least one of the hydrogens is replaced with halogen (—CH₂X, —CHX₂ or —CX₃).

In the present invention, “aralkyl” is the generic term for —(CH₂)ₙAr, which is a moiety in which aryl substitutes for a carbon of alkyl. Examples of the aralkyl include benzyl (—CH₂C₆H₅) and phenethyl (—CH₂CH₂C₆H₅).

In the present invention, aryl is, unless defined otherwise, an unsaturated aromatic ring including a single ring, or multiple rings (preferably, 1 to 4 rings) conjugated or connected by a covalent bond. Non-limiting examples of the aryl include phenyl, biphenyl, o-terphenyl, m-terphenyl, p-terphenyl, 1-naphthyl, 2-naphthyl, 1-anthryl, 2-anthryl, 9-anthryl, 1-phenanthrenyl, 2-phenanthrenyl, 3-phenanthrenyl, 4-phenanthrenyl, 9-phenanthrenyl, 1-pyrenyl, 2-pyrenyl, and 4-pyrenyl.

In the present invention, heteroaryl is a moiety in which one or more carbon atoms in the aryl defined above are substituted with a non-carbon atom such as nitrogen, oxygen or sulfur. Non-limiting examples of the heteroaryl include furyl, tetrahydrofuryl, pyrrolyl, pyrrolidinyl, thienyl, tetrahydrothienyl, oxazolyl, isoxazolyl, triazolyl, thiazolyl, isothiazolyl, pyrazolyl, pyrazolidinyl, oxadiazolyl, thiadiazolyl, imidazolyl, imidazolinyl, pyridyl, pyridaziyl, triazinyl, piperidinyl, morpholinyl, thiomorpholinyl, pyrazinyl, piperainyl, pyrimidinyl, naphthyridinyl, benzofuranyl, benzothienyl, indolyl, indolinyl, indolizinyl, indazolyl, quinolizinyl, quinolinyl, isoquinolinyl, cinnolinyl, phthalazinyl, quinazolinyl, quinoxalinyl, pteridinyl, quinuclidinyl, carbazoyl, acridinyl, phenazinyl, phenothizinyl, phenoxazinyl, purinyl, benzimidazolyl, benzothiazolyl, and analogs conjugated therewith.

In the present invention, unless defined otherwise, a hydrocarbon ring (cycloalkyl) or a hydrocarbon ring having a hetero atom (heterocycloalkyl) may be understood as a cyclic structure of an alkyl or heteroalkyl, respectively.

Non-limiting examples of the cycloalkyls include cyclopentyl, cyclohexyl, 1-cyclohexenyl, 3-cyclohexenyl, and cycloheptyl. Non-limiting examples of the heterocycloalkyls include 1-(1,2,5,6-tetrahydropyrinyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothiene-2-yl, tetrahydrothiene-3-yl, 1-piperazinyl, and 2-piperazinyl.

In addition, the cycloalkyl or heterocycloalkyl may have a form in which cycloalkyl, heterocycloalkyl, aryl or heteroaryl is conjugated or connected by a covalent bond.

Here, the polyalkyleneoxide is a water-soluble polymer moiety, and examples of such polyalkyleneoxides include polyethylene glycol (PEG), polypropylene glycol (PPG), a polyethylene glycol-polypropylene glycol (PEG-PPG) copolymer, and N-substituted methacrylamide-containing polymer and copolymer.

The polyalkyleneoxide may be additionally substituted, as needed, as long as the characteristics of the polymer are maintained. For example, the substitution may be a chemical bond for increasing or decreasing the chemical or biological stability of a polymer. As a specific example, any carbon or terminal carbon in the polyalkyleneoxide may be substituted with hydroxy, alkyl ether (methyl ether, ethyl ether, propyl ether or the like), carboxylemethyl ether, carboxyethyl ether, benzyl ether, dibenzylmethyl ether, or dimethylamine. In one embodiment, the polyalkyleneoxide may be polyethyleneoxide terminated with methyl ether (mPEG), wherein mPEG is represented by the formula —(CH₂CH₂O)ₙCH₃, whose size may change depending on the size of n corresponding to the number of ethylene glycol repeat units.

In addition, the reporter represented by Formula 1, 2, 5 or 6 may have a counter ion. The counter ion, which is an organic or inorganic anion, may be suitably selected in consideration of the solubility and stability of the reporter.

Examples of the counter ions of the reporter according to one embodiment of the present invention include inorganic anions such as a phosphoric acid hexafluoride ion, a halogen ion, a phosphoric acid ion, a perchloric acid ion, a periodic acid ion, an antimony hexafluoride ion, a tartaric acid hexafluoride ion, a fluoroboric acid ion, and a tetrafluoride ion; and organic anions such as a thiocyanate ion, a benzenesulfonic acid ion, a naphthalenesulfonic acid ion, a p-toluenesulfonic acid ion, an alkylsulfonic acid ion, a benzenecarboxylic acid ion, an alkylcarboxylic acid ion, a trihaloalkylcarboxylic acid ion, an alkyl sulfonic acid ion, a trihaloalkylsulfonic acid ion, and a niconinic acid ion. In addition, metal compound ions such as bisphenylditol, thiobisphenol chelate, and bisdiol-α-diketone, metal ions such as sodium and potassium, and quaternary ammonium salts may also be selected as the counter ions.

Specific examples of the reporters represented by Formulas 1, 2, 5 and 6 are as follows. However, the following exemplary compounds are provided to help understanding the reporters defined herein, but not limit the scope of the reporters defined herein. The following exemplary compounds may be synthesized by a known synthesis method with reference to preparation examples described herein, or the contents defined herein regarding to the reporters represented by Formulas 1, 2, 5 and 6.

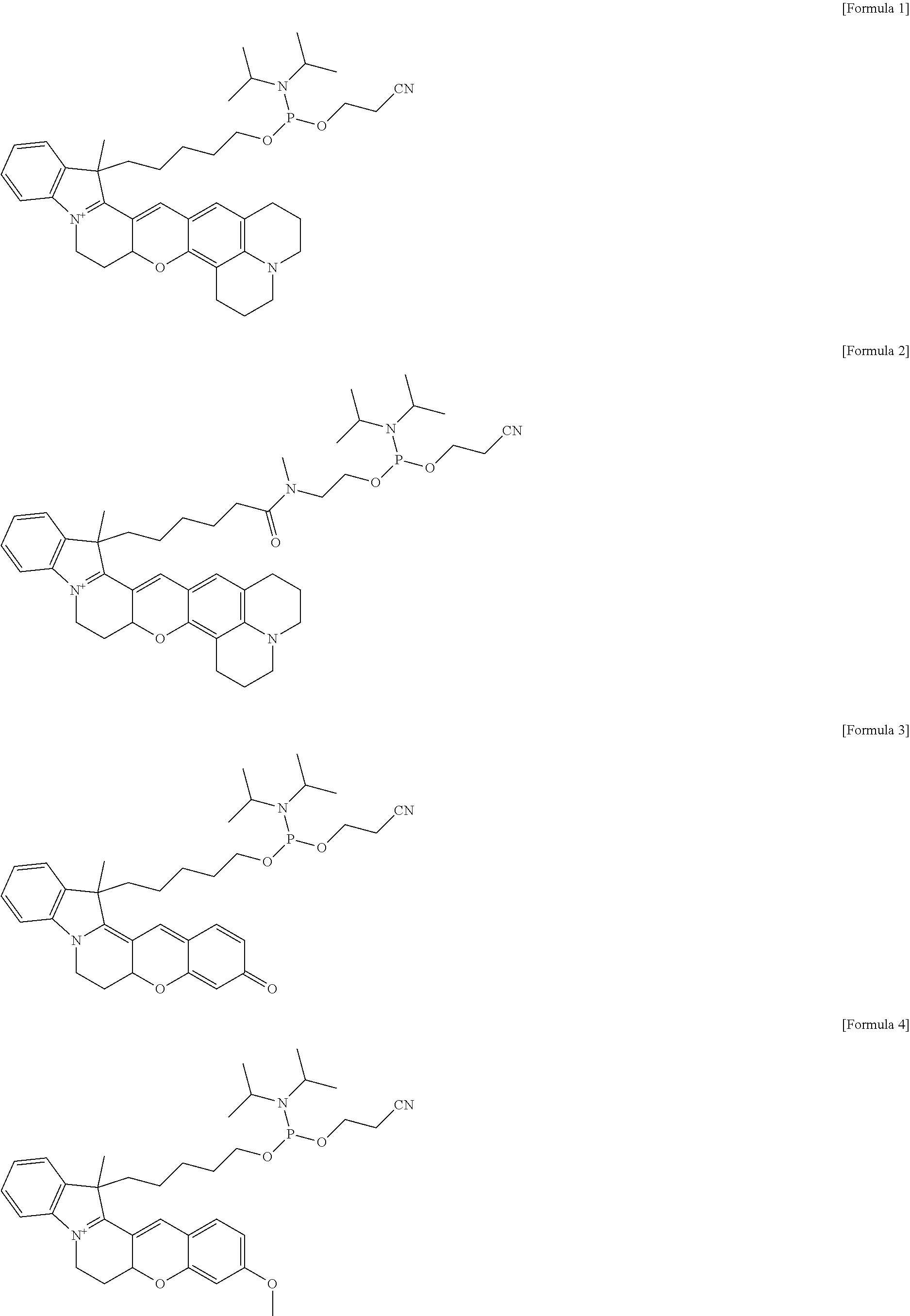 [SMILES: CC(C)N(C(C)C)P(OCCC#N)OCCCCCC1(C)C2=[N+](CCC3Oc4c(cc5c6c4CCCN6CCC5)C=C23)c2ccccc21 ; CC(C)N(C(C)C)P(OCCC#N)OCCN(C)C(=O)CCCCCC1(C)C2=[N+](CCC3Oc4c(cc5c6c4CCCN6CCC5)C=C23)c2ccccc21 ; CC(C)N(C(C)C)P(OCCC#N)OCCCCCC1(C)C2=C3C=C4C=CC(=O)C=C4OC3CCN2c2ccccc21 ; COc1ccc2c(c1)OC1CC[N+]3=C(C1=C2)C(C)(CCCCCOP(OCCC#N)N(C(C)C)C(C)C)c1ccccc13]

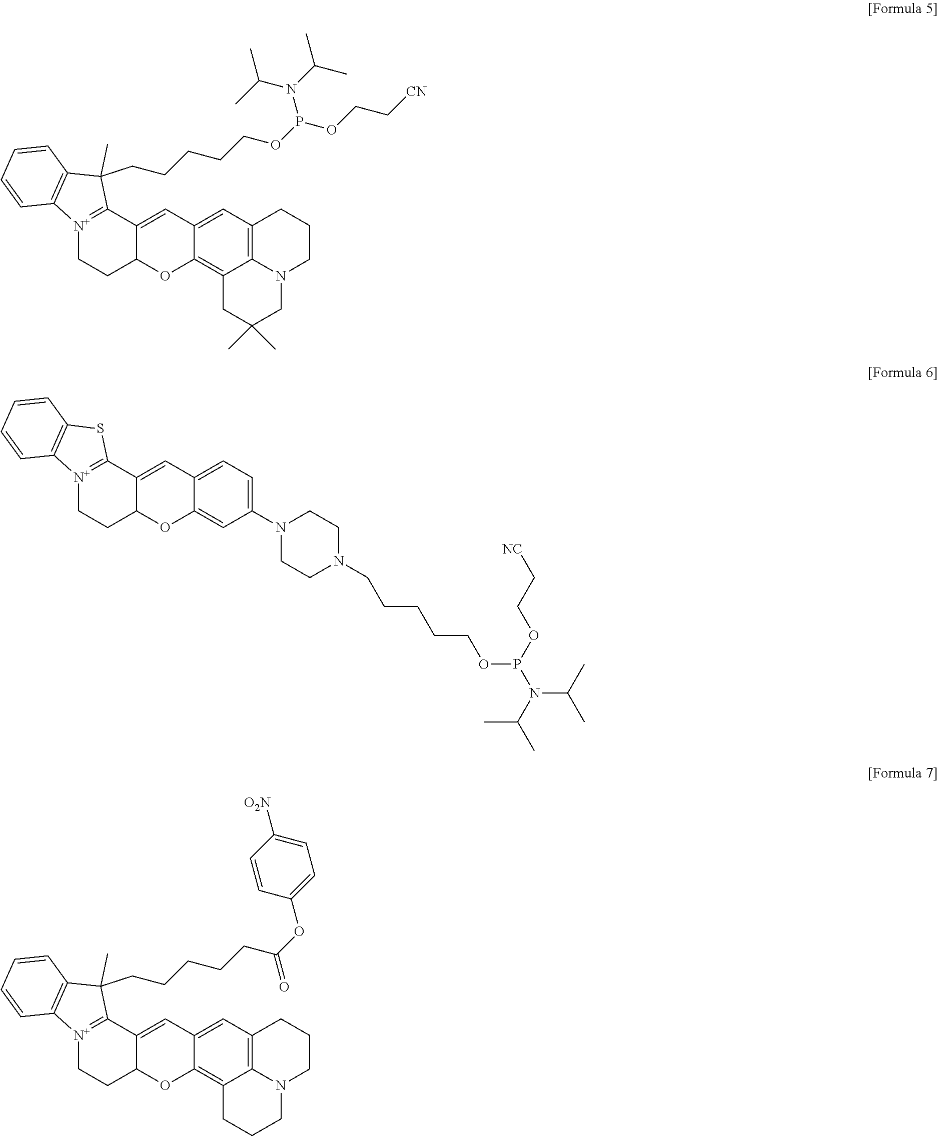 [SMILES: CC(C)N(C(C)C)P(OCCC#N)OCCCCCC1(C)C2=[N+](CCC3Oc4c(cc5c6c4CC(C)(C)CN6CCC5)C=C23)c2ccccc21 ; CC(C)N(C(C)C)P(OCCC#N)OCCCCCN1CCN(c2ccc3c(c2)OC2CC[n+]4c(sc5ccccc54)C2=C3)CC1 ; CC1(CCCCCC(=O)Oc2ccc([N+](=O)[O-])cc2)C2=[N+](CCC3Oc4c(cc5c6c4CCCN6CCC5)C=C23)c2ccccc21]

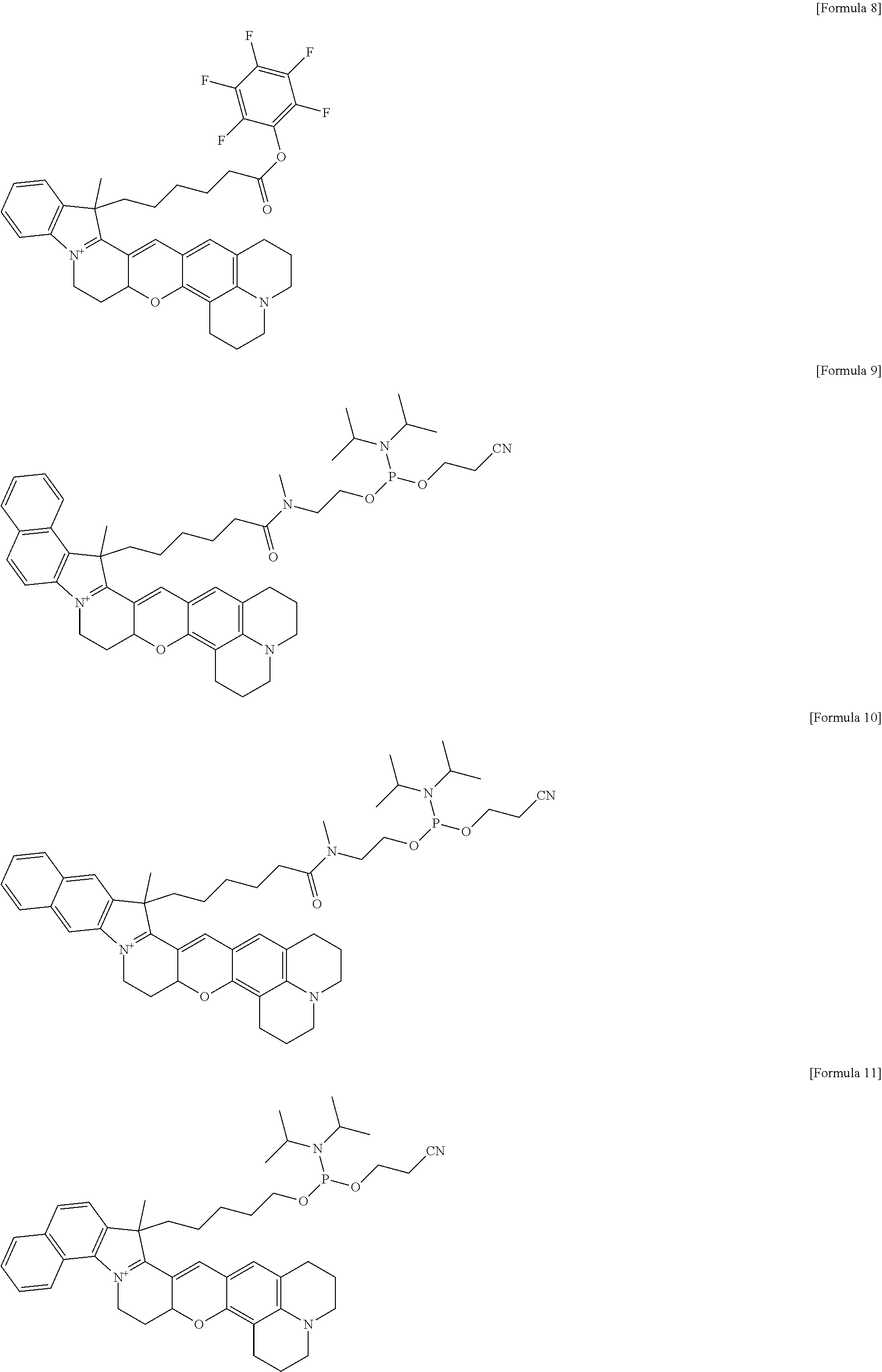 [SMILES: CC1(CCCCCC(=O)Oc2c(F)c(F)c(F)c(F)c2F)C2=[N+](CCC3Oc4c(cc5c6c4CCCN6CCC5)C=C23)c2ccccc21 ; CC(C)N(C(C)C)P(OCCC#N)OCCN(C)C(=O)CCCCCC1(C)C2=[N+](CCC3Oc4c(cc5c6c4CCCN6CCC5)C=C23)c2ccc3ccccc3c21 ; CC(C)N(C(C)C)P(OCCC#N)OCCN(C)C(=O)CCCCCC1(C)C2=[N+](CCC3Oc4c(cc5c6c4CCCN6CCC5)C=C23)c2cc3ccccc3cc21 ; CC(C)N(C(C)C)P(OCCC#N)OCCCCCC1(C)C2=[N+](CCC3Oc4c(cc5c6c4CCCN6CCC5)C=C23)c2c1ccc1ccccc21]

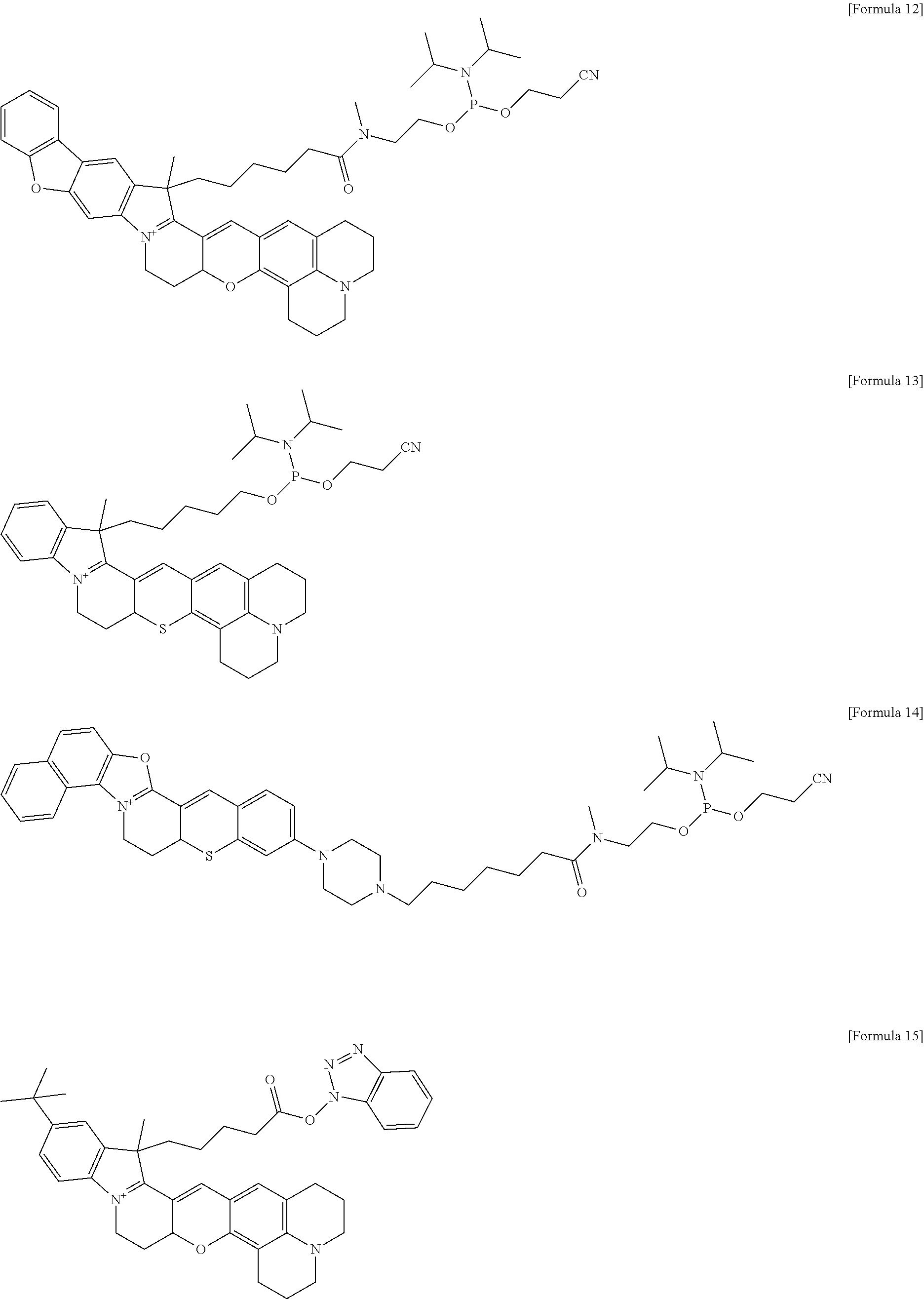 [SMILES: CC(C)N(C(C)C)P(OCCC#N)OCCN(C)C(=O)CCCCCC1(C)C2=[N+](CCC3Oc4c(cc5c6c4CCCN6CCC5)C=C23)c2cc3oc4ccccc4c3cc21 ; CC(C)N(C(C)C)P(OCCC#N)OCCCCCC1(C)C2=[N+](CCC3Sc4c(cc5c6c4CCCN6CCC5)C=C23)c2ccccc21 ; CC(C)N(C(C)C)P(OCCC#N)OCCN(C)C(=O)CCCCCCN1CCN(c2ccc3c(c2)SC2CC[n+]4c(oc5ccc6ccccc6c54)C2=C3)CC1 ; CC(C)(C)c1ccc2c(c1)C(C)(CCCCC(=O)On1nnc3ccccc31)C1=[N+]2CCC2Oc3c(cc4c5c3CCCN5CCC4)C=C12]

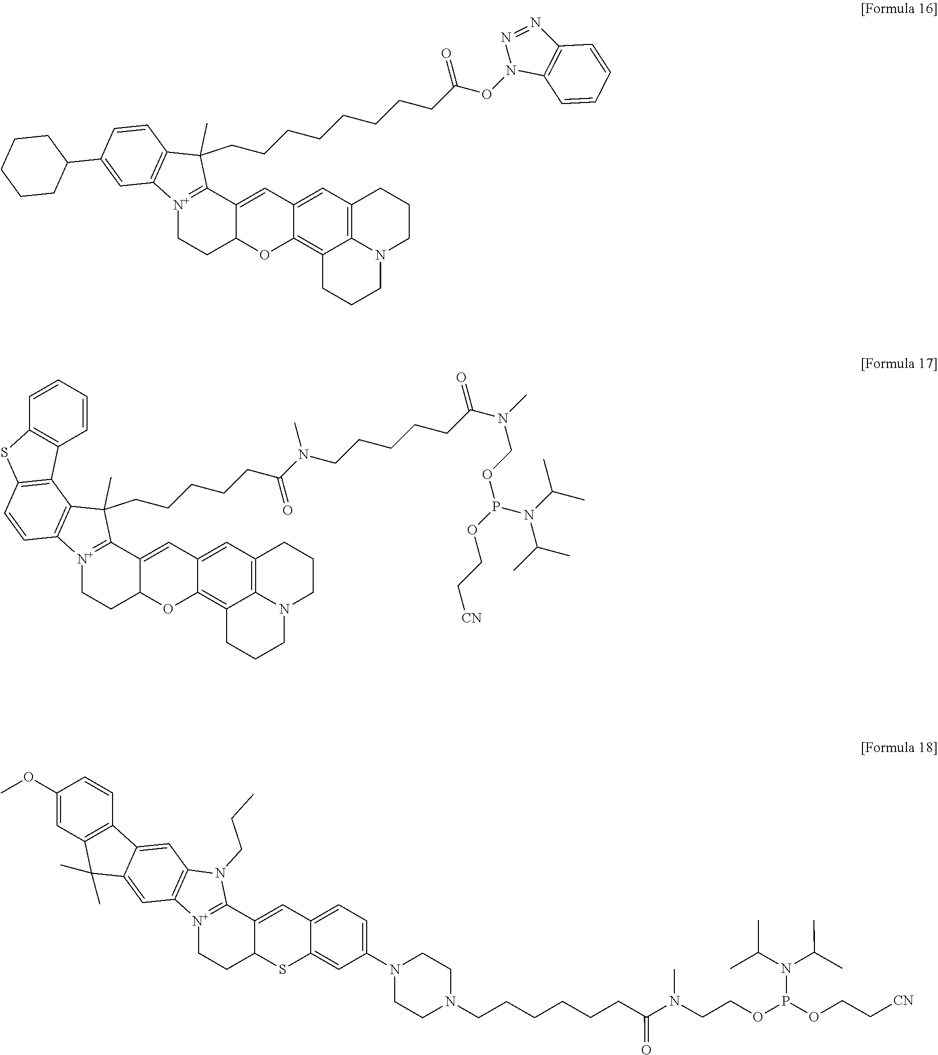

A biomolecule targeted by the reporter represented by Formula 1, 2, 5 or 6 disclosed herein may be at least one selected from an antibody, a lipid, a protein, a peptide, a carbohydrate, a nucleic acid (including DNA, RNA or a nucleotide), and preferably, a nucleic acid (including DNA, RNA or a nucleotide).

Specific examples of lipids include fatty acids, phospholipids, and lipopolysaccharides, and specific examples of carbohydrates include monosaccharides, disaccharides, and polysaccharides (e.g., dextran).

Here, a biomolecule may include at least one selected from amino, sulphydryl, carbonyl, hydroxyl, carboxyl, phosphate and thiophosphate, or a derivative thereof as any moiety of the reporter represented by Formula 1, 2, 5 or 6 or a moiety for reacting with a reactive group binding to the reporter represented by Formula 1, 2, 5 or 6.

In addition, a biomolecule may be an oxy or dioxy polynucleic acid which includes at least one selected from amino, sulphydryl, carbonyl, hydroxyl, carboxyl, phosphate and thiophosphate, or a derivative thereof.

Moreover, other than a biomolecule, the reporter represented by Formula 1, 2, 5 or 6 may be used to label a drug a hormone (including a receptor ligand), a receptor, an enzyme or an enzyme substrate, cells, a cell membrane, a toxin, a microorganism or a nano bio material (a polystyrene microsphere, etc.) including at least one selected from amino, sulphydryl, carbonyl, hydroxyl, carboxyl, phosphate and thiophosphate.

Oligonucleotide, Composition for Detecting Nucleic Acid, and Support for Detecting Nucleic Acid, Including Novel Reporter

According to another aspect of the present invention, an oligonucleotide including at least one selected from the reporters represented by Formulas 1, 2, 5, and 6 is provided.

The oligonucleotide refers to a polymer of 1 to hundreds of nucleotides, and includes all of DNA, RNA, and PNA. In addition, examples of such oligonucleotides include those that can be easily modified by one of ordinary skill in the art, such as analogs thereof, for example, those in which chemical modifications have been applied to the nucleotides, or those in which sugars are linked, and mean single-stranded or double-stranded ones.

The oligonucleotide preferably includes a probe. Such a probe is more preferably a probe complementarily bound with a target nucleic acid, but the present invention is not limited thereto. Here, the probe may be selected from a nucleic acid, a peptide, a saccharide, an oligonucleotide, a protein, an antibody, or a combination thereof, but the present invention is not limited thereto.

In one embodiment, the oligonucleotide may include a quencher. For example, the 5′ end of the oligonucleotide may be labeled with the reporter represented by Formula 1, 2, 5 or 6, and the 3′ end thereof may be labeled with the quencher. A probe complementarily binding to a target nucleic acid may be located between the 5′ end and the 3′ end.

The maximum absorbance of the quencher available in the present invention may be 620 to 700 nm, and preferably, 660 to 680 nm, and the absorbance range of the quencher may be 530 to 730 nm. In addition, the maximum absorbance and absorbance range of the quencher may be appropriately selected considering the fluorescent property of the reporter defined herein.

It is important that the probe is designed such that the reporter can be sufficiently quenched by the quencher while minimizing signal crosstalk. Accordingly, when designing a probe, it is necessary to confirm that the reporter and the quencher, which are attached to the 5′ end and the 3′ end of the probe for labeling according to the type of target biomolecule (e.g., a nucleic acid), are compatible with each other.

As the quencher, various known or compatible quenchers (e.g., BHQ0, BHQ1, BHQ2, BHQ3, BBQ650, DABCYL, TAMRA, MGBEclipse, Atto540Q, Atto575Q, Atto612Q, QSY7, and QSY21) may be used. In addition, as the quencher, the quenchers disclosed in Korean Unexamined Patent Application Publication No. 10-2020-0067733 may be used. Representative examples of the quenchers disclosed in Korean Unexamined Patent Application Publication No. 10-2020-0067733 are as follows.

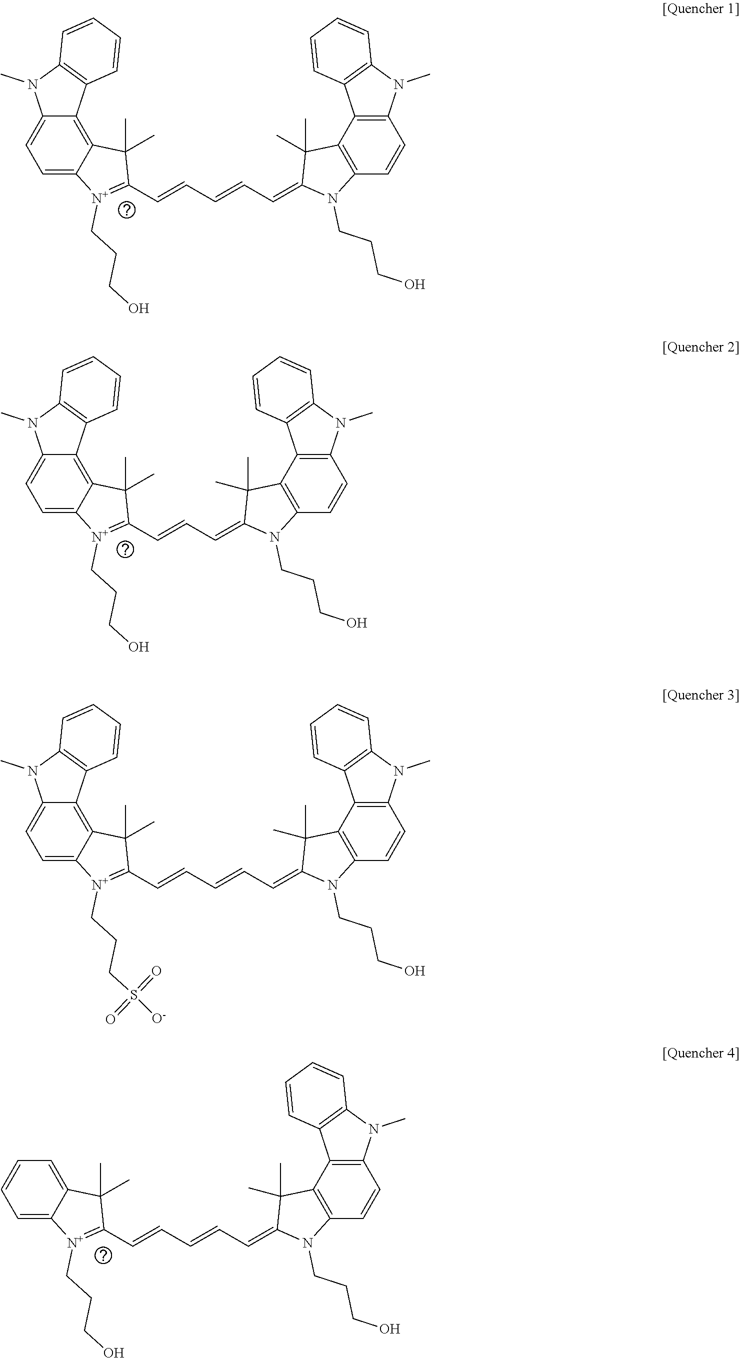

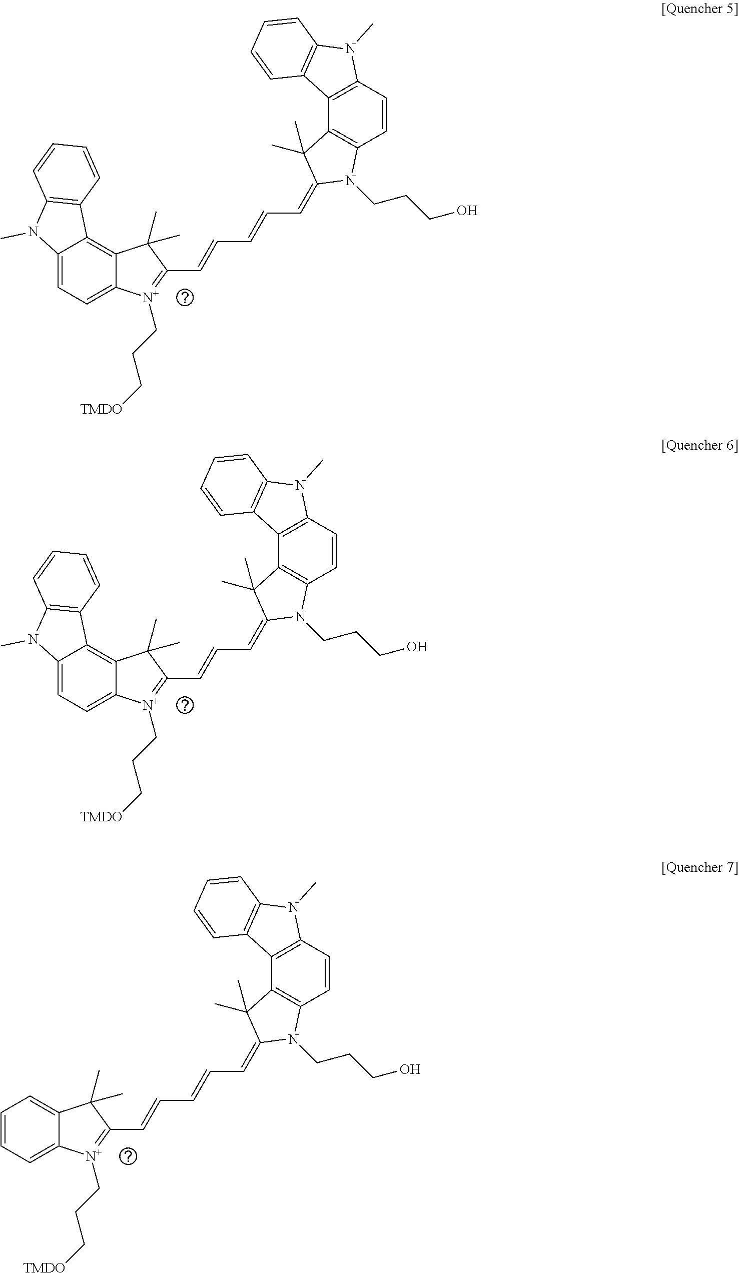

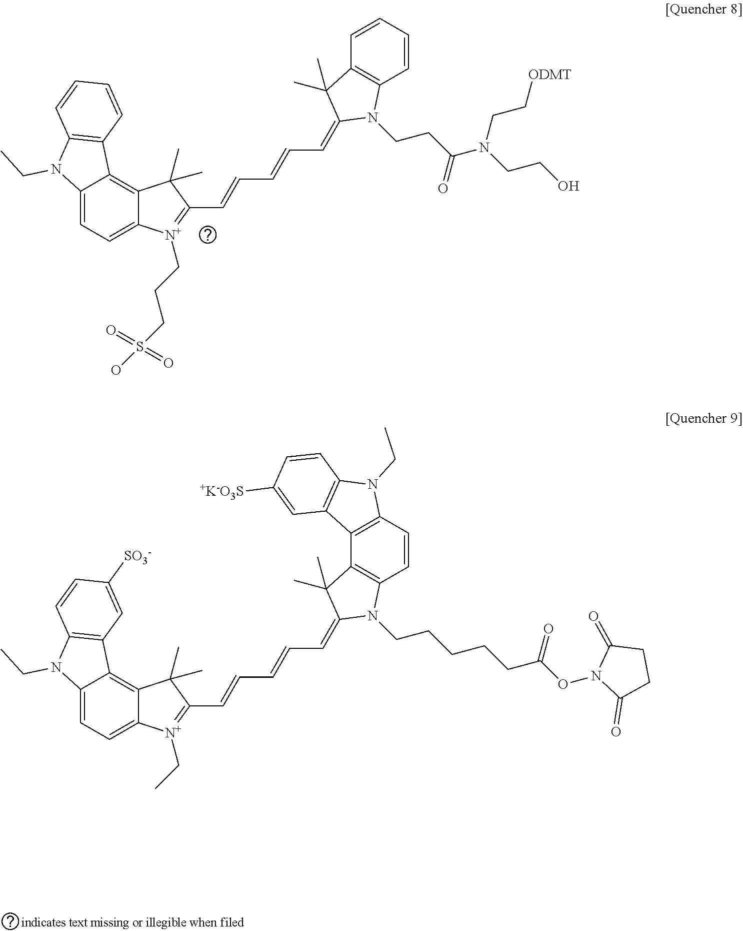

In addition, the oligonucleotide according to the present invention may further include a minor groove binder (MGB) to improve a binding strength with a nucleic acid.

The MGB is a crescent-shaped probe that can selectively make a non-covalent bond to a minor groove (e.g., shallow furrow in the DNA helix) included in a nucleic acid such as DNA.

Such an oligonucleotide may be used in various ways in the fields of chemistry and biology. Particularly, it may be useful for real time PCR or microassay, but the present invention is not limited thereto.

In addition, according to another aspect of the present invention, a composition for detecting a nucleic acid, including the oligonucleotide, is provided.

The composition for detecting a nucleic acid according to one embodiment of the present invention may further include an enzyme, a solvent (buffer, etc.) and other reagents, which are used for a reaction with a target biomolecule, in addition to an oligonucleotide including the reporter represented by Formula 1, 2, 5 or 6, MGB and a quencher at the same time.

Here, as the solvent, a buffer selected from the group consisting of a phosphate buffer, a carbonate buffer and a Tris buffer, an organic solvent selected from dimethyl sulfoxide, dimethylformamide, dichloromethane, methanol, ethanol and acetonitrile, or water may be used, and it is possible to adjust solubility by introducing various moieties to the reporter according to the type of solvent.

In addition, according to still another aspect of the present invention, a support for detecting a nucleic acid, which includes the reporter represented by Formula 1, 2, 5 or 6, a support, and a linker that connects the reporter and the support, is provided.

Accordingly, a biomolecule in a sample may be fixed on a support through interaction with the reporter adhered onto the support.

Here, rather than the reporter represented by Formula 1, 2, 5 or 6 only being connected to the support via the linker, a probe dual-labeled with the reporter represented by Formula 1, 2, 5 or 6 and a quencher may be connected.

Here, as described above, the probe may have a form in which the reporter represented by Formula 1, 2, 5 or 6 may be attached to the 5′ end of the probe, and the quencher may be attached to the 3′ end, and a probe that can complementarily bind to a target nucleic acid may be located between the 5′ end and the 3′ end.

The support may be manufactured with at least one selected from glass (e.g., controlled pore glass (CPG)), cellulose, nylon, acrylamide gel, dextran, polystyrene, a resin, alginate, collagen, peptides, fibrin, hyaluronic acid, agarose, polyhydroxyethylmethacrylate, polyvinyl alcohol, polyethylene glycol, polyethyleneoxide, polyethylene glycol diacrylate, gelatin, Matrigel, polylactic acid, carboxymethylcellulose, chitosan, latex, and Sepharose, but the present invention is not limited thereto. Also, the support may be formed as beads or a membrane.

Here, the linker is a part connecting the reporter and the support, and any material capable of connecting the reporter and the support may be used as a linker intended by the present invention.

For example, the linker may be selected from substituted or unsubstituted C₁-C₃₀ alkyl, substituted or unsubstituted C₃-C₃₀ cycloalkyl, substituted or unsubstituted C₂-C₃₀ heteroalkyl having at least one hetero atom, substituted or unsubstituted C₂-C₃₀ heterocycloalkyl having at least one hetero atom, substituted or unsubstituted C₂-C₃₀ alkenyl, substituted or unsubstituted C₆-C₃₀ aryl, substituted or unsubstituted C₃-C₃₀ heteroaryl, amide (—CONH—), ester (—COO—), ketone (—CO—), nucleoside, and any combination thereof.

Examples of the connecting structures between the supports and the quenchers mediated by the linkers are as follows.

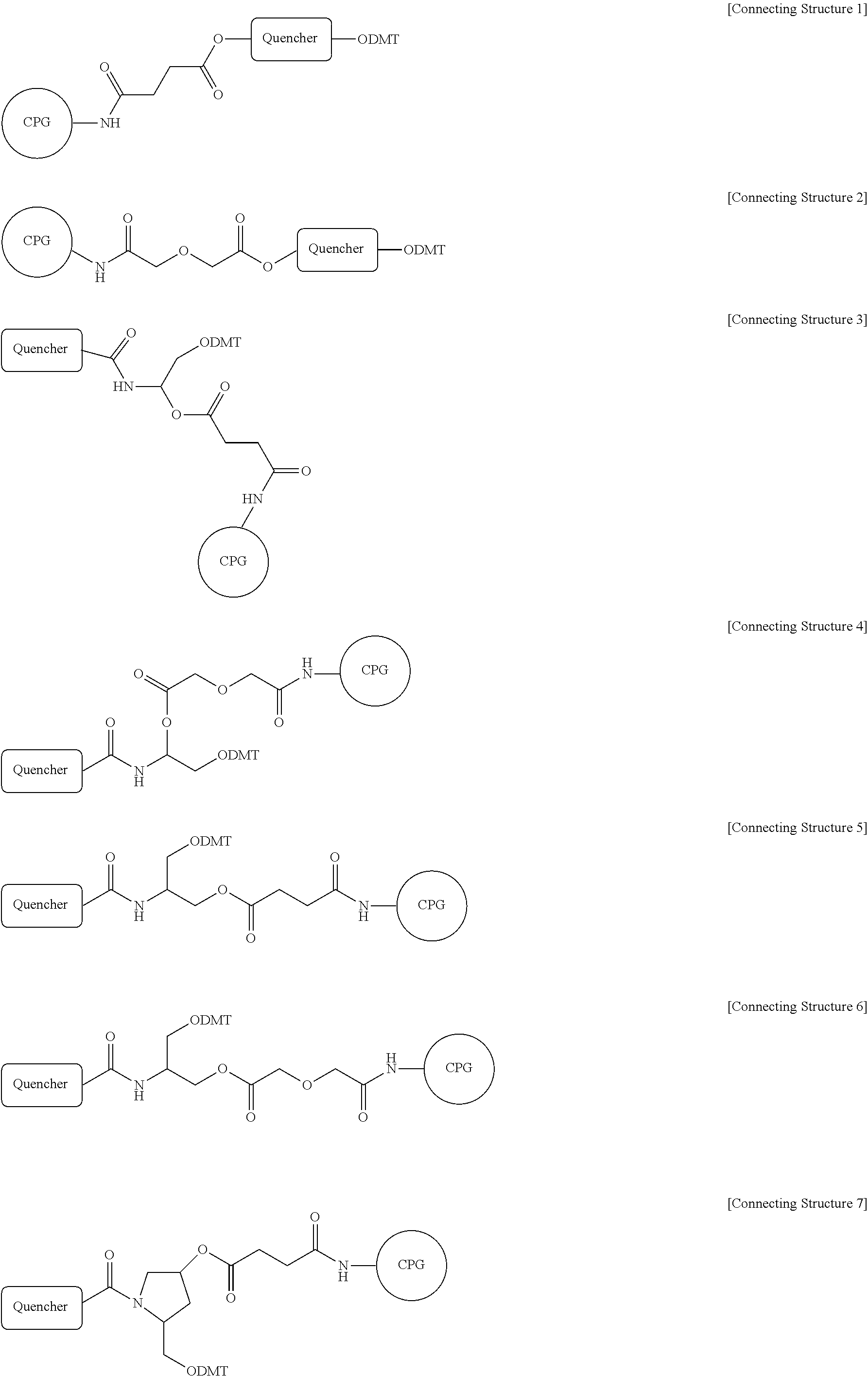

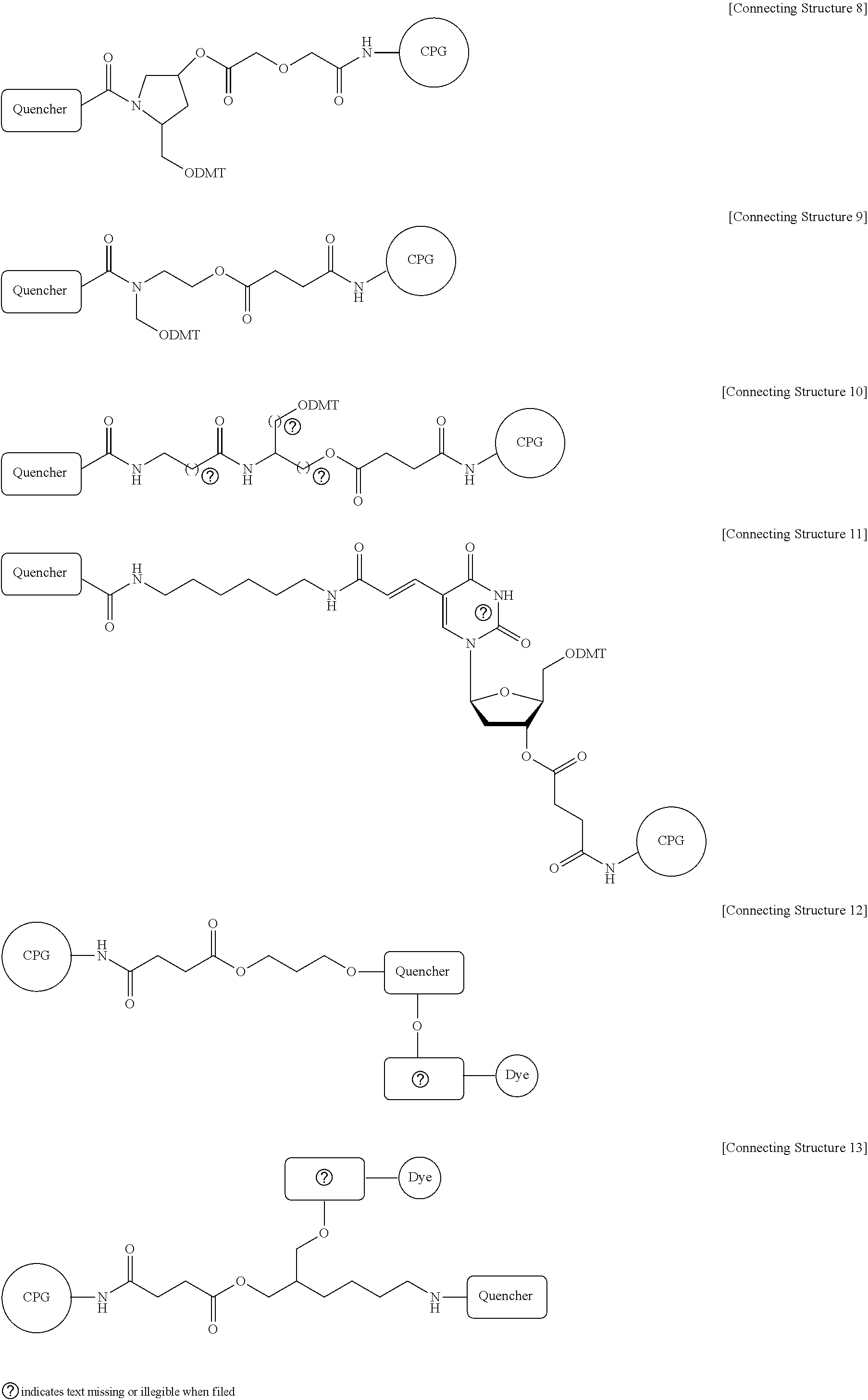

The linker merely connects a reporter and a support, but does not affect any other reaction of a reporter or fluorophore, or fluorescence or quenching actions.

Method of Detecting Nucleic Acid

According to one embodiment of the present invention, a method of labeling a target nucleic acid through reaction with a reporter-labeled probe or a probe double labeled with a reporter and a quencher may be implemented. In addition, a method of labeling a biomolecule using a target-specific interaction by introducing an appropriate reactive group to a reporter according to the type of target biomolecule may be implemented. In addition, a method of identifying the biomolecule labeled with the reporter through electrophoresis may be implemented.

DNA Microarray

A DNA microarray is for measuring the fluorescence of a target nucleic acid by preparing a single-stranded probe nucleic acid which labels a target nucleic acid through reaction with a dye and has a complementary base sequence to the target nucleic acid, and hybridizing the probe nucleic acid with the target nucleic acid denatured into a single strand on a substrate.

In the labeling method, when gene expression is investigated, as the probe nucleic acid immobilized to the substrate, cDNA, which is prepared by amplifying a cDNA library, genome library, or any of all genomes as a template through PCR, may be used.

In addition, for investigation of a gene mutation, various oligonucleotides corresponding to mutations may be synthesized based on a known sequence serving as a reference and used.

A proper method for immobilizing the probe nucleic acid on the substrate may be selected according to the type of nucleic acid or substrate. For example, a method for electrostatic bonding to a substrate surface-treated with a cation such as polylysine using the charge of DNA may also be used.

The target nucleic acid denatured into a single strand is immobilized on the substrate, and hybridized with the oligonucleotide. Here, the 5′ end of the oligonucleotide is labeled with a fluorophore, and the 3′ end thereof is labeled with at least one selected from the reporters represented by Formulas 1, 2, 5 and 6. Between the 5′ end and the 3′ end, a probe that is able to complimentarily bind to the targeted nucleic acid may be located.

Hybridization is preferably performed at room temperature to 70° C. for approximately 2 to 48 hours. Through hybridization, the target nucleic acid having a complementary base sequence with the probe nucleic acid is selectively bound with the probe nucleic acid. Afterward, the substrate is washed and dried at room temperature.

Here, the oligonucleotide is hybridized to the target nucleic acid by the probe, but the fluorophore at the 5′ end is present in a quenched state by the quencher at the 3′ end.

Subsequently, the oligonucleotide hybridized to the target nucleic acid is elongated by a polymerase, and separated from the target nucleic acid by the exonuclease activity of the polymerase and degraded. The fluorophore at the 5′ end of the oligonucleotide and the quencher at the 3′ end thereof are separated from each other, and thus the fluorophore may exhibit fluorescence.

Here, the intensity of the generated fluorescence is measured to measure the amplification amount of the target nucleic acid.

PCR Method

According to a PCR method, a probe complementary to the base sequence of a target nucleic acid to be labeled is labeled with a reporter, and reacted with the target nucleic acid before or after the amplification of the target nucleic acid, followed by measuring fluorescence of the target nucleic acid.

Specifically, the elongation reaction of the target nucleic acid is carried out by an enzyme (DNA polymerase or RNA polymerase), and here, a double-stranded nucleic acid sequence formed of the target nucleic acid and a primer consisting of an oligonucleotide is recognized by the enzyme to accomplish the elongation reaction from the recognition site, and only a target gene area is amplified.

When synthesis is performed by the enzyme, the synthesis reaction is accomplished using nucleotides (dNTP and NTP) as raw materials.

Here, by mixing common nucleotides (dNTP and NTP) with reporter-bearing nucleotides in an arbitrary ratio, a nucleic acid into which the equivalent amount of a dye is introduced may be synthesized.

In addition, a nucleic acid into which a reporter is introduced may be synthesized by bonding the reporter after introducing a nucleotide having an amino group in an arbitrary ratio by PCR.

When synthesis is performed by the enzyme, the synthesis reaction is accomplished using nucleotides as raw materials, and here, when a material in which OH at the 3′ end of the nucleotide is substituted with H is used, a nucleic acid is no longer elongated, and at this point of time, the reaction ends.

The nucleotides, that is, dideoxy nucleotide triphosphate (ddNTP) are called a terminator.

When a terminator is mixed with common nucleotides to synthesize a nucleic acid, the terminator is introduced with certain probability and thus the reaction ends, so nucleic acids of various lengths are synthesized.

When these are separated by size through gel electrophoresis, DNA is sequenced in order of length. Here, when being labeled with a different reporter for each type of base of the terminator, at the end point (3′ end) of the synthesis reaction, a dependency on each base is observed, on the basis of the reporter attached to the terminator, fluorescence data is read, thereby obtaining sequencing data of the target nucleic acid.

In addition, instead of the terminator, primers previously labeled with the reporter may be used to hybridize with a target nucleic acid.

In addition, as a probe, a peptide nucleic acid (PNA) may also be used. PNA is obtained by replacing the pentose phosphate backbone, which is the basic skeleton of a nucleic acid, with a polyamide backbone composed of glycine as a unit, and PNA has a 3D structure highly similar to the nucleic acid, and is very specific for a nucleic acid having a complementary base sequence and strongly binds thereto. Accordingly, PNA may also be used as a reagent for telomere research by applying a telomere PNA probe, in addition to a convention DNA analysis method such as in-situ hybridization (ISH).

For labeling, for example, double-stranded DNA is brought into contact with PNA having a base sequence complementary to all or a part of the base sequence of DNA and labeled with the reporter to hybridize, the mixture is heated to generate single-stranded DNA, and slowly cooled to a room temperature to prepare a PNA-DNA complex, followed by measuring fluorescence.

In this example, a method of measuring fluorescence of a product by amplifying a target nucleic acid through PCR has been described, but according to this method, it is necessary to investigate the amount of the amplification product by checking the size of the product through electrophoresis and then measuring a fluorescence intensity.

To this end, the amount of the product may be measured in real time using a probe designed to generate fluorescence by hybridizing an energy transfer of a fluorescent dye to the PCR product.

For example, DNA labeled with a donor and an acceptor may be used. A specific labeling method may be a molecular beacon method, TaqMan-PCR, or a cycling probe method, which is used to confirm the presence of a nucleic acid of a specific sequence.

Other Labeling Methods

In addition, the reporter of the present invention may also be used in a method of labeling a target using specific bonding.

That is, in the labeling of a sample including a target or a sample modified by a modifying material, one of a binding material specifically binding the sample and a binding material specifically binding to a modifying material may be labeled with a reporter, and fluorescence may be measured from the labeled binding materials.

Here, for the combination of the sample or modifying material with the binding material, antigen-antibody, hapten-anti-hapten antibody, biotin-avidin, a Tag antigen, a Tag antibody, lectin-glycoprotein, or hormone-receptor may be used.

Specifically, in terms of an antigen present in a substrate, solution, beads, or an antibody, a specific antigen may be labeled through antigen-specific interaction of an antibody by reacting with a binding material such as a reporter-labeled antibody.

An antigen may be a protein, a polysaccharide, a nucleic acid, or a peptide, and other than the antigen, a hapten such as a low-molecular-weight molecule, for example, FITC or a dinitrophenyl group may also be used. Here, as an antigen (or hapten)-antibody combination, GFP, anti GFP antibody, FITC, or anti-FITC antibody is used.

The labeled antigens may be used in various measurement methods including immunostaining, ELISA, Western blotting or flow cytometry.

In addition, using the reporter of the present invention, an intracellular signaling phenomenon may also be observed. Various enzymes are involved in internal signaling or cell responses according to the signaling. In a representative signaling phenomenon, it is known that a special protein kinase is activated, thereby inducing protein phosphorylation to initiate signaling.

Binding and hydrolysis of a nucleotide (e.g., ATP or ADP) play a critical role in its activity, and an intracellular signaling phenomenon may be observed with high sensitivity by introducing a reporter into a nucleotide derivative.

In addition, the reporter of the present invention may also be used in observation of a gene expression phenomenon using RNA interfering (RNAi).

RNAi is inhibition of expression by degradation of mRNA of a target gene by introducing double-stranded RNA (dsRNA) into cells, and it is possible to observe the RNAi phenomenon by labeling the designed dsRNA with a reporter.

In addition, since having a reactive group capable of labeling a target nucleic acid or target protein in tissue or cells, the reporter of the present invention may be used as a dye for confirming a transcription level of the target nucleic acid or an expression level of the target protein.

Hereinafter, specific examples of the present invention are suggested. However, the following examples are only for exemplifying or explaining the present invention in detail, and the present invention should not be limited thereto. In addition, among the reporters defined in the claims and detailed description of the present invention, compounds whose synthesis methods are not disclosed through the following preparation examples may be synthesized with reference to the following preparation examples.

### Preparation Example 1. Synthesis of Compound 1

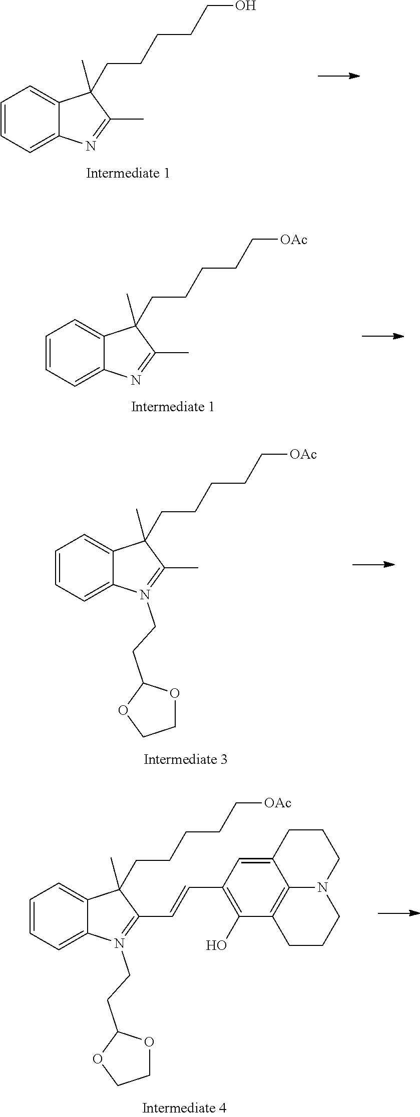

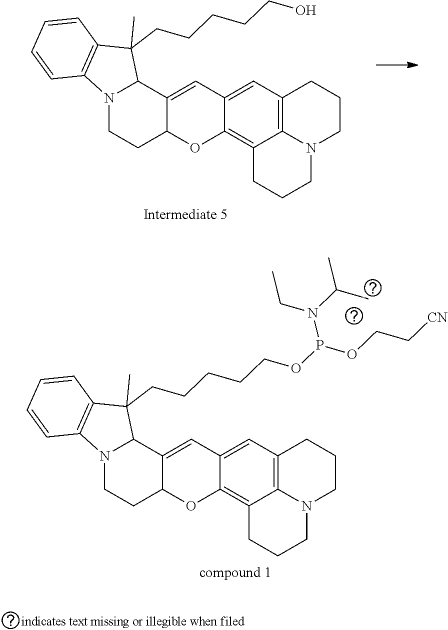 [SMILES: CC1(CCCCCO)c2ccccc2N2CCC3Oc4c(cc5c6c4CCCN6CCC5)C=C3C21 ; CCN(C(C)C)P(OCCC#N)OCCCCCC1(C)c2ccccc2N2CCC3Oc4c(cc5c6c4CCCN6CCC5)C=C3C21]

### Synthesis of Intermediate 2

Intermediate 1 (synthesized with reference to International laid-open Patent Application Publication No. 2004-039894; 26 g, 0.113 mmol), triethylamine (34 g, 0.341 mmol), acetic anhydride (23 g, 0.227 mol), N, N-dimethylaminopyridine (1.4 g, 0.0113 mol), and dichloromethane (300 mL) were put into a 1 L 1-neck reactor, followed by stirring at room temperature for 2 hours. Water (300 mL) was put into the reactor and vigorously stirred to separate an organic layer. After adding anhydrous magnesium sulfate and stirring for 5 minutes, the solid was filtered, and the filtrate was concentrated.

### Synthesis of Intermediate 3

Intermediate 2 (29 g, 0.107 mol), 2-(2-bromoethyl)-1,3-dioxolane (29 g, 0.160 mol), and potassium iodide (35 g, 0.213 mol) were added in acetonitrile (300 mL), and refluxed and stirred for 12 hours in a 500 mL 1-neck reactor. After cooling, the solid was filtrated, and the filtrate was concentrated and purified through a column.

### Synthesis of Intermediate 4

Intermediate 3 (33 g, 0.0668 mol), 8-hydroxyjulolidine-9-carboxaldehyde (13 g, 0.0601 mol), and ethanol (300 mL) were put into a 500 mL 1-neck reactor and refluxed and stirred for 12 hours, followed by cooling and concentration.

### Synthesis of Intermediate 5

Intermediate 4 (46 g, 0.0668 mol), a 50% sulfuric acid solution (90 mL) and chloroform (470 mL) were put into a 1 L 1-neck reactor, and stirred at room temperature for 30 minutes. Water (300 mL) was put into the reactor and vigorously stirred to separate an organic layer. After adding anhydrous sodium sulfate and stirring for 5 minutes, the solid was filtered, and the filtrate was concentrated and purified through a column.

### Synthesis of Compound 1

Intermediate 5 (13 g, 0.0219 mol), 2-cyanoethyl N,N-diisopropylchloro phosphoramidite (6.7 g, 0.0285 mol), triethylamine (34 g, 0.341 mmol), and dichloromethane (260 mL) were put into a 500 mL 1-neck reactor and stirred at room temperature for 30 minutes. Water (300 mL) was put into the reactor and vigorously stirred to separate an organic layer. After adding anhydrous sodium sulfate and stirring for 5 minutes, the solid was filtered, and the filtrate was concentrated and purified through a column.

¹H-NMR of the obtained Compound 1 is as follows.

¹H-NMR (300 MHz, CDCl₃) δ 7.72-7.71 (m, 1H), 7.46-7.32 (m, 4H), 6.93 (d, 1H, J=11.1 Hz), 5.64-5.50 (m, 1H), 4.65-4.42 (m, 2H), 3.83-3.70 (m, 2H), 3.55-3.42 (m, 8H), 3.10-2.90 (m, 1H), 2.81-2.54 (m, 6H), 2.41-2.09 (m, 2H), 1.80-1.66 (m, 3H), 1.48-1.08 (m, 16H), 0.97-0.94 (m, 1H), 0.68-0.53 (m, 1H)

### Preparation Example 2. Synthesis of Compound 2

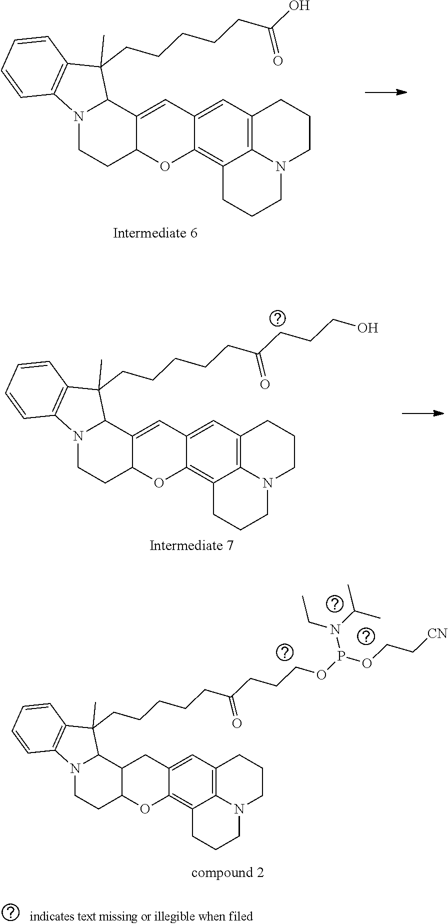 [SMILES: CC1(CCCCCC(=O)O)c2ccccc2N2CCC3Oc4c(cc5c6c4CCCN6CCC5)C=C3C21 ; CC1(CCCCCC(=O)CCCO)c2ccccc2N2CCC3Oc4c(cc5c6c4CCCN6CCC5)C=C3C21 ; CCN(C(C)C)P(OCCC#N)OCCCC(=O)CCCCCC1(C)c2ccccc2N2CCC3Oc4c(cc5c6c4CCCN6CCC5)CC3C21]

### Synthesis of Intermediate 7

Intermediate 6 (synthesized with reference to Korean Unexamined Patent Application Publication No. 10-2017-0009795) (3.5 g, 5.41 mmol), 2-(methylamino)ethanol (0.4 g, 5.41 mmol), N-methylmorpholine (1.6 g, 16.24 mmol), HATU (CAS No. 148893-10-1) (2.4 g, 6.49 mmol), and dimethylformamide (35 mL) were put into a 250 mL 1-neck reactor, stirred at room temperature for 1 hour, followed by concentration and purification through a column.

### Synthesis of Compound 2

Intermediate 7 (2.2 g, 3.14 mmol), 2-cyanoethyl N,N-diisopropylchloro phosphoramidite (0.8 g, 3.458 mol), triethylamine (0.8 g, 7.86 mmol), and dichloromethane (45 mL) were put into a 250 mL 1-neck reactor, and stirred at room temperature for 30 minutes. Water (50 mL) was put into the reactor and vigorously stirred to separate an organic layer. After adding anhydrous sodium sulfate and stirring for 5 minutes, the solid was filtered, and the filtrate was concentrated and purified through a column.

¹H-NMR (300 MHz, CDCl₃) δ 7.72-7.70 (m, 1H), 7.48-7.26 (m, 4H), 6.95-6.90 (m, 1H), 5.36-5.29 (m, 1H), 4.43-4.08 (m, 2H), 3.86-3.65 (m, 4H), 3.63-3.44 (m, 8H), 3.02-2.90 (m, 3H), 2.80-2.56 (m, 7H), 2.47-2.05 (m, 1H), 2.29-1.97 (m, 8H), 1.73-1.65 (m, 3H), 1.50-1.12 (m, 16H), 0.98-0.87 (m, 1H), 0.67-0.57 (m, 1H)

### Preparation Example 3. Synthesis of Double-Labeled Probe (Oligtonucleotide)

Using Universal UnyLinker Support (Chemgene, 500 Å), each of 5′-GCGGGAGATGATATGGACTT-3′ as a forward primer for black queen cell virus (BQCV) and 5′-CCGTCTGAGATGCATGAATAC-3′ as a reverse primer was synthesized at 1 μmol scale.

First, a dual-labeled probe was synthesized by synthesizing 5′-CCATCTTTATCGGTACGCCGCCC-quencher-3′ as a single-labeled probe (synthesized with reference to Korean Unexamined Patent Application Publication No. 10-2020-0067733) using quencher-CPG, and attaching Compound 1 and either Cal Fluor© Red 610, Texas Red® or ROX, which is commercialized fluorescent material, as reporters to the 5′ end of 5′-CCATCTTTATCGGTACGCCGCCC-quencher-3′ using a MerMade™ 48×DNA synthesizer.

The forms of the synthesized dual-labeled probes are shown in Table 1 below.

TABLE 1

| Classification | Dual-labeled probe |
|---|---|
| Example 1 | 5′-Compound 1-Probe-Quencher-3′ |
| Comparative Example 1 | 5′- Cal Fluor ® Red 610-Probe-Quencher-3′ |
| Comparative Example 2 | 5′- Texas Red ®-Probe-Quencher-3′ |
| Comparative Example 3 | 5′-ROX-Probe-Quencher-3′ |

The structure of the quencher-CPG used in Preparation Example 3 is shown below.

[symbol]

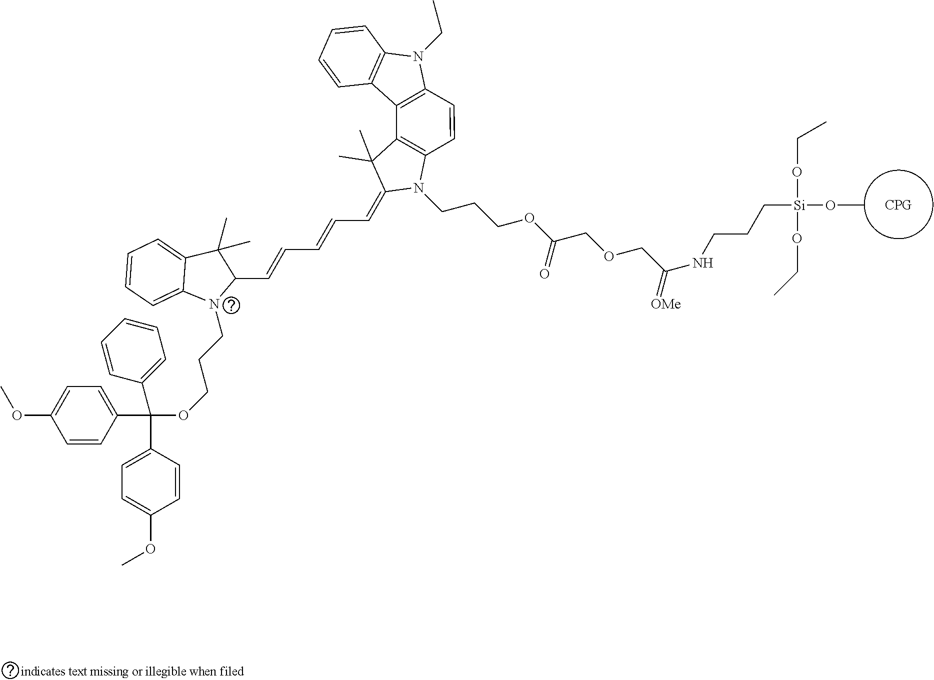

The absorption/emission wavelength ranges of Compound 1, Cal Fluor® Red 610, Texas Red® and ROX, used as reporters in Preparation Example 3, are shown in Table 2 below, and the absorption wavelength range of a quencher is shown in Table 3 below.

TABLE 2

| Reporter | Excitationₘₐₓ (nm) | Emissionₘₐₓ (nm) |
|---|---|---|
| Compound 1 | 602 | 628 |
| Cal Fluor ® Red 610 | 590 | 610 |
| Texas Red ® | 595 | 615 |
| ROX | 588 | 608 |

TABLE 3

| Classification | λ_{Max}(nm) | ε (mol⁻¹ · cm⁻¹) |
|---|---|---|
| Quencher | 660-680 | 140,000 |

### Preparation Example 4. Synthesis of Dual-Labeled Probe (Oligonucleotide)

Using Universal UnyLinker Support (Chemgene, 500 Å), each of 5′-ATGCAACATTAACCCGAGATACG-3′ as a forward primer for Chlamydia trachomatis (CT) and 5′-ACTCGGCTTGGGAAGAGCTT-3′ as a reverse primer was synthesized at a 1 μmol scale.

First, a dual-labeled probe was synthesized by synthesizing 5′-TTGTCCATATCTTTGATACGACGCCGC-quencher-3′ as a single-labeled probe (synthesized with reference to Korean Unexamined Patent Application Publication No. 10-2020-0067733) using quencher-CPG (the same as that used in Preparation Example 3), and attaching Compound 2, and either Cal Fluor© Red 610, Texas Red® or ROX, which is a commercialized fluorescent material, as reporters to the 5′ end of 5′-TTGTCCATATCTTTGATACGACGCCGC-quencher-3′ using a MerMade™ 48×DNA Synthesizer.

The forms of the synthesized dual-labeled probes are shown in Table 4 below.

TABLE 4

| Classification | Dual-labeled probe |
|---|---|
| Example 2 | 5′-Compound 2-Probe-Quencher-3′ |
| Comparative Example 1 | 5′- Cal Fluor ® Red 610-Probe-Quencher-3′ |
| Comparative Example 2 | 5′- Texas Red ®-Probe-Quencher-3′ |
| Comparative Example 3 | 5′-ROX-Probe-Quencher-3′ |

The absorption/emission wavelength ranges of Compound 2 used as a reporter in Preparation Example 4 are shown in Table 5 below.

TABLE 5

| Reporter | Excitationₘₐₓ (nm) | Emissionₘₐₓ (nm) |
|---|---|---|
| Compound 2 | 602 | 628 |

### Experimental Example 1. Real-Time PCR Experiment Using Dual-Labeled Probe

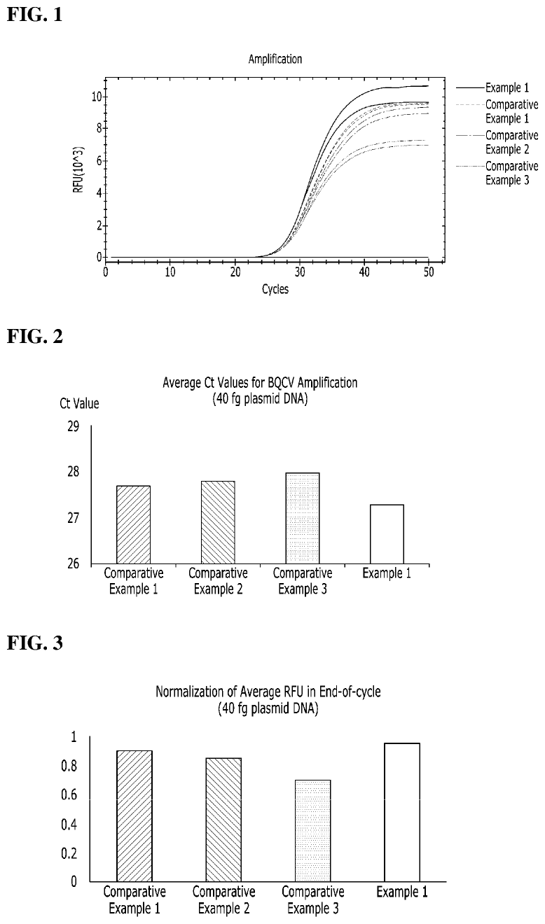

Real-time PCR was repeatedly performed twice for BQCV plasmid DNA with a composition shown in Table 6 below using each dual-labeled probe synthesized according to Preparation Example 3 (using CFX-96, Biorad). The results of real-time PCR are shown in FIGS. 1 to 3.

TABLE 6

| Classification | Content (μl) |
|---|---|
| (Enzynomics)TOPreal ™ qPCR 2X PreMIX | 10 |
| (TaqMan Probe) | |
| gDNA (10 ng/μl) | 1 |
| Beta-actin F/R MIX (5 pmole/μl) | 1 |
| Dual-labeled probe (3 pmole/μl) | 3 |
| DEPC water | 5 |

### Experimental Example 2. Real-Time PCR Experiment Using Dual-Labeled Probe

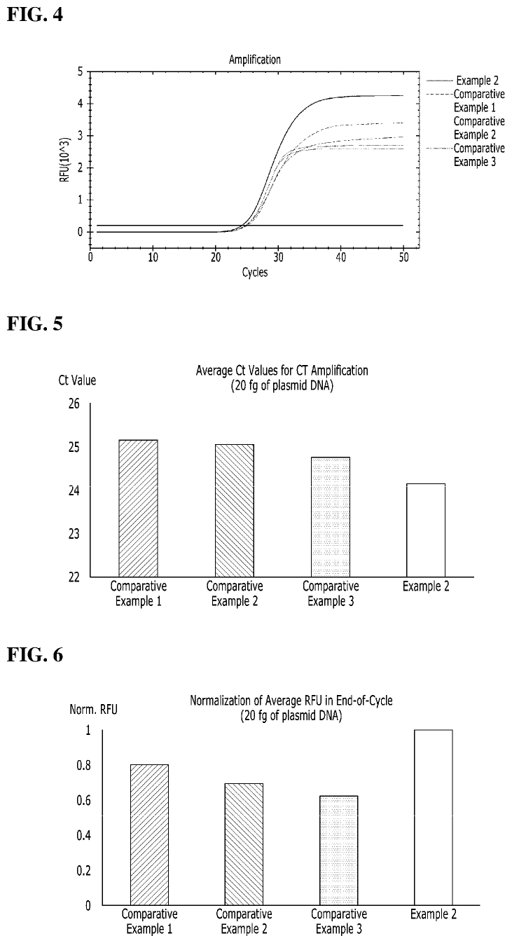

Real-time PCR was repeatedly performed twice on Chlamydia trachomatis (CT) plasmid DNA with a composition shown in Table 7 using a dual-labeled probe synthesized according to Preparation Example 4 (using CFX-96, Biorad). The results of real-time PCR are shown in FIGS. 4 to 6.

TABLE 7

| Classification | Content (μl) |
|---|---|
| (Bioline)SensiFAST ™ Probe No-ROX Mix (2X) | 10 |
| CT plasmid DNA (20 fg/μl) | 1 |
| CT F/R primer mix (10 pmole/μl) | 1 |
| CT Dual-labeled probe (5 pmole/μl) | 3 |
| DEPC water | 5 |

Referring to the real-time PCR results, considering that average Ct and RFU values of real-time PCR using dual-labeled probes in which Compounds 1 and 2, and commercialized fluorescent materials Cal Fluor© Red 610, Texas Red® and ROX are included as reporters, it can be confirmed that almost similar PCR efficiencies are shown.

However, it can be confirmed that the dual-labeled probes using Compound 1 or 2 as a reporter shows a relatively low Ct value, and has a higher finally amplified RFU value, compared to other commercially available fluorescent materials.

Considering a molecular diagnosis aspect, since the limit of detection (LoD) of Compound 1 or 2 is lower than a conventional commercialized fluorescent material, when Compound 1 or 2 is used as a reporter of a dual-labeled probe, even though target DNA or RNA in a sample is present at a relatively low concentration, it is shown that the ease of detection of Compound 1 or 2 can be higher than other commercialized fluorescent materials.

Accordingly, a reporter for labeling a nucleic acid defined herein may be applied to conventional nucleic acid labeling and detection fields (e.g., a PCR experiment, etc.), or may sufficiently replace a conventional commercialized fluorescent material.

Although embodiments of the present invention have been described above, it will be understood by those of ordinary skill in the art that the present invention may be modified and altered in various ways by adding, altering, or deleting a component without departing from the spirit of the present invention defined in the appended claims, and such modifications and alterations will also be included in the scope of the present invention.

What is claimed is:

1. A reporter for labeling a nucleic acid, represented by Formula 1 or 2 below: 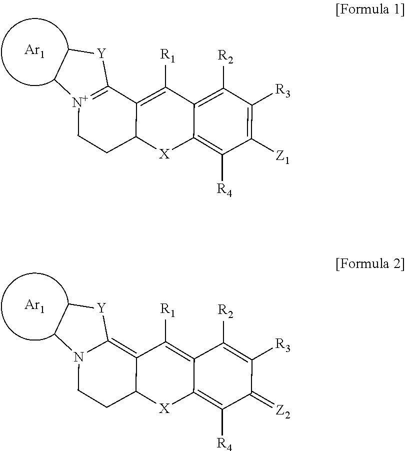
Wherein,
Ar₁ is substituted or unsubstituted C₆-C₂₀ aryl or C₆-C₂₀ heteroaryl including at least one hetero atom,
Z₁ is NR₅R₆ or OR₇,
Z₂ is NR₈ or O,
X is O or S,
Y is CR₉R₁₀, NR₁₁, O or S,
R₁ to R₁₁ are each independently a moiety selected from hydrogen, deuterium, substituted or unsubstituted C₁-C₁₀ alkyl, substituted or unsubstituted C₁-C₁₀ heteroalkyl including at least one hetero atom, substituted or unsubstituted C₂-C₁₀ alkenyl, substituted or unsubstituted C₂-C₁₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O—), substituted or unsubstituted C₁-C₁₀ alkoxy, substituted or unsubstituted C₃-C₂₀ cycloalkyloxy, substituted or unsubstituted C₅-C₂₀ aryloxy, substituted or unsubstituted C₂-C₂₀ heteroaryloxy, substituted or unsubstituted C₅-C₂₀ aryl, substituted or unsubstituted C₂-C₂₀ heteroaryl, substituted or unsubstituted C₅-C₂₀ aralkyl, substituted or unsubstituted C₁-C₁₀ alkylthio, substituted or unsubstituted C₅-C₂₀ arylthio, substituted or unsubstituted C₃-C₂₀ cycloalkylthio, substituted or unsubstituted C₂-C₂₀ heteroarylthio, substituted or unsubstituted acylamino, acyloxy, substituted or unsubstituted phosphino, carboxylate (—CO₂—), trifluoromethylsulfonyl (—SO₂CF₃), substituted or unsubstituted ammonium, nitro, sulfonic acid (—SO₃H), sulfonate, substituted sulfonyl, substituted sulfonic acid ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkyleneoxide, or each independently forms a ring by binding adjacent two moieties to each other, and
at least one of R₁ to R₁₁ is a moiety substituted with phosphoramidite or active ester.

2. The reporter of claim 1, wherein the reporter is represented by Formula 1,
Z₁ is NR₅R₆, and
R₅ or R₆ is bound with R₃ or R₄ to form a ring.

3. The reporter of claim 1, wherein the reporter is represented by Formula 1,
Z₁ is OR₇, and
R₇ is bound with R₃ or R₄ to form a ring.

4. The reporter of claim 1, wherein the reporter is represented by Formula 2,
Z₂ is NR₈, and
R₈ is bound with R₃ or R₄ to form a ring.

5. The reporter of claim 1, wherein the active ester is selected from a N-hydroxysuccinimide derivative, a hydroxybenzotriazole derivative, a 1-hydroxy-7-azabenzotriazole derivative, a nitrophenol derivative, and a pentafluorophenol derivative.

6. The reporter of claim 1, wherein at least one of R₁ to R₁₁ is a moiety whose end is substituted with phosphoramidite or active ester.

7. The reporter of claim 1, wherein at least one of R₁ to R₁₁ is any moiety substituted with the moiety represented by Formula 3 below: 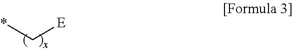
Wherein,
* indicates a position to which the moiety binds,
x is an integer between 2 to 10, and
E is phosphoramidite or active ester.

8. The reporter of claim 1, wherein at least one of R₁ to R₁₁ is any moiety substituted with the moiety represented by Formula 4 below: 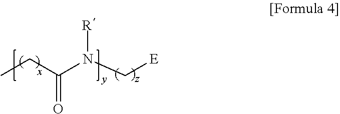
Wherein,
* indicates a position to which the moiety binds,
x is an integer between 2 to 10,
y is an integer between 1 to 5,
z is an integer between 0 to 5,
R′ is hydrogen, deuterium, or substituted or unsubstituted C₁-C₁₀ alkyl, and
E is phosphoramidite or active ester.

9. The reporter of claim 1, wherein Y is CR₉R₁₀, and
at least one of R₉ and R₁₀ is a moiety substituted with phosphoramidite or active ester.

10. The reporter of claim 1, which is represented by Formula 5 or 6 below: 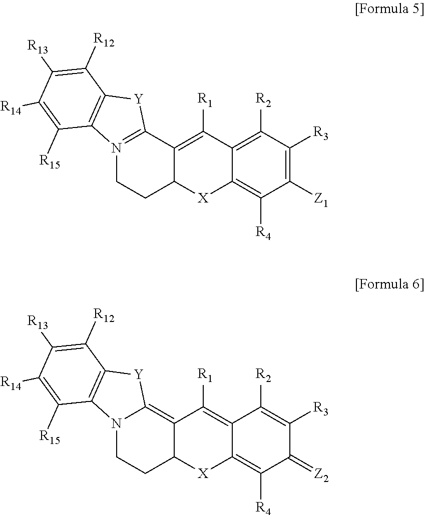
Wherein,
Z₁ is NR₅R₆ or OR₇,
Z₂ is NR₈ or O,
X is O or S,
Y is CR₉R₁₀, NR₁₁, O or S,
R₁ to R₁₅ are each independently a moiety selected from hydrogen, deuterium, substituted or unsubstituted C₁-C₁₀ alkyl, substituted or unsubstituted C₁-C₁₀ heteroalkyl including at least one hetero atom, substituted or unsubstituted C₂-C₁₀ alkenyl, substituted or unsubstituted C₂-C₁₀ alkynyl, substituted or unsubstituted C₃-C₂₀ cycloalkyl, substituted or unsubstituted C₃-C₂₀ cycloalkenyl, substituted or unsubstituted C₂-C₂₀ heterocycloalkyl, hydroxy, oxido (—O—), substituted or unsubstituted C₁-C₁₀ alkoxy, substituted or unsubstituted C₃-C₂₀ cycloalkyloxy, substituted or unsubstituted C₅-C₂₀ aryloxy, substituted or unsubstituted C₂-C₂₀ heteroaryloxy, substituted or unsubstituted C₅-C₂₀ aryl, substituted or unsubstituted C₂-C₂₀ heteroaryl, substituted or unsubstituted C₅-C₂₀ aralkyl, substituted or unsubstituted C₁-C₁₀ alkylthio, substituted or unsubstituted C₅-C₂₀ arylthio, substituted or unsubstituted C₃-C₂₀ cycloalkylthio, substituted or unsubstituted C₂-C₂₀ heteroarylthio, substituted or unsubstituted acylamino, acyloxy, substituted or unsubstituted phosphino, carboxylate (—CO₂—), trifluoromethylsulfonyl (—SO₂CF₃), substituted or unsubstituted ammonium, nitro, sulfonic acid (—SO₃H), sulfonate, substituted sulfonyl, substituted sulfonic acid ester, substituted or unsubstituted sulfonamide, substituted thioketone, trihalomethyl (—CF₃, —CCl₃, —CBr₃, or —CI₃), haloformyl (—COCl, —COBr, or —COI), formyl (—CHO), acyl, carboxyl, substituted ester, substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, substituted or unsubstituted silyl, substituted or unsubstituted amide, carbamate, carboxylate, substituted or unsubstituted phosphine, substituted or unsubstituted phosphoric acid, phosphate, phosphonic acid, phosphonate, nitrile, hydrazine, acetal, ketal, and polyalkyleneoxide, or each independently forms a ring by binding adjacent two moieties to each other, and
at least one of R₁ to R₁₅ is a moiety substituted with phosphoramidite or active ester.

11. An oligonucleotide comprising:
the reporter of claim 1; and
a quencher.

12. The oligonucleotide of claim 11, further comprising a minor groove binder (MGB) interposed between the reporter and the quencher.

13. A composition for detecting a nucleic acid, comprising the oligonucleotide of claim 11.

14. A support for detecting a nucleic acid, comprising:
the reporter of claim 1, or a probe which is dual-labeled with the reporter of claim 1 and a quencher;
a support; and
a linker connecting the reporter or probe with the support.

15. The support of claim 14, wherein the support is glass, cellulose, nylon, acrylamide, gel, dextran, polystyrene, or resin.

16. The support of claim 14, wherein the linker is selected from substituted or unsubstituted C₁-C₃₀ alkyl, substituted or unsubstituted C₃-C₃₀ cycloalkyl, substituted or unsubstituted C₂-C₃₀ heteroalkyl having at least one hetero atom, substituted or unsubstituted C₂-C₃₀ heterocycloalkyl having at least one hetero atom, substituted or unsubstituted C₂-C₃₀ alkenyl, substituted or unsubstituted C₆-C₃₀ aryl, substituted or unsubstituted C₃-C₃₀ heteroaryl, amide (—CONH—), ester (—COO—), ketone (—CO—), nucleoside, and any combination thereof.

17. A method of detecting a nucleic acid, comprising:
(a) preparing a reaction mixture including a target nucleic acid, a reagent necessary for amplifying the target nucleic acid, and the oligonucleotide of claim 11;
(b) amplifying the target nucleic acid in the reaction mixture by a polymerase chain reaction; and
(c) measuring a fluorescence intensity of the reaction mixture.

18. The method of claim 17, wherein (b) comprises
(b-1) elongating an oligonucleotide hybridized to the target nucleic acid by a polymerase;
(b-2) separating a reporter and a quencher of the oligonucleotide from the target nucleic acid by the exonuclease activity of the polymerase; and
(b-3) emitting fluorescence by the reporter separated from the quencher.

19. The method of claim 17, further comprising:
(d) measuring an amplification amount of the target nucleic acid from the fluorescence intensity measured in (c).
